(19) United States
(12) Patent Application Publication
(10) Pub. No.: US 2026/0212493 A1
(43) Pub. Date: Jul. 23, 2026

(54) DEVICES, SYSTEMS, AND METHODS FOR ARTIFACT REMOVAL IN MULTIMODAL IMAGING

(71) Applicants: Institute of Science Tokyo, Tokyo (JP); Canon Medical Systems Corporation, Tochigi (JP)

(72) Inventors: Taishi Yonetsu, Tokyo (JP); Seiji Takeuchi, Newton, MA (US); Kohei Watanabe, Utsunomiya (JP); Manabu Tanaka, Otawara-shi (JP)

(21) Appl. No.: 19/034,381

(22) Filed: Jan. 22, 2025

(51) Int. Cl. G06T 7/00; A61B 5/00; G06T 5/70; G06T 7/70

(52) U.S. Cl. CPC G06T 7/0012; A61B 5/0066; A61B 5/0071; G06T 5/70; G06T 7/70; G06T 2207/10024; G06T 2207/10064; G06T 2207/10101

(57) ABSTRACT

Devices, systems, and methods obtain imaging data, wherein the imaging data include at least fluorescence-imaging data; detect artifact data in the fluorescence-imaging data; generate modified fluorescence-imaging data by removing the artifact data or by changing the artifact data in the fluorescence-imaging data to a specified value; and generate an image based on the modified fluorescence-imaging data.

### BACKGROUND

Field: This application generally concerns devices, systems, and methods that perform imaging (e.g., medical imaging) using multiple imaging modalities.

Background: Bendable optical-imaging devices (e.g., endoscopes, flexible borescopes) enable the imaging of internal tissues, organs, and structures. For example, in cardiology, a bendable optical-imaging device that is capable of optical coherence tomography (OCT) may be used to acquire depth-resolved images of a sample (e.g., tissues, organs). Additionally, some bendable optical-imaging devices use fluorescence imaging, such as near-infrared fluorescence (“NIRF”) (e.g., near-infrared autofluorescence (“NIRAF”)). Fluorescence imaging enables the visualization of molecular processes (e.g., biological processes in an organism). The bendable optical-imaging device, which may include a flexible body, a coil, and an optical probe, may be navigated through a lumen (e.g., a vessel) or a cavity.

### BRIEF DESCRIPTION OF THE DRAWINGS

FIG. 1 is a schematic of an example embodiment of a medical-imaging system.

FIG. 2 is a schematic of an example embodiment of the medical-imaging system of FIG. 1 that illustrates additional details of the probe-interface subsystem.

FIGS. 3A-B illustrate an example embodiment of a bendable optical-imaging device.

FIG. 4 is a schematic of an example embodiment of the medical-imaging system of FIG. 1 that illustrates additional details of the probe-interface subsystem.

FIG. 5 illustrates an example embodiment of a screen from a user interface.

FIG. 6 illustrates an example embodiment of an operational flow for reducing or eliminating artifacts in images that are generated from fluorescence-detection data.

FIG. 7A illustrates an example embodiment of an operational flow for removing or replacing an artifact in fluorescence-detection data.

FIG. 7B illustrates an example embodiment of an operational flow for removing or replacing an artifact in fluorescence-detection data.

FIG. 8A illustrates an example embodiment of a set of fluorescence images that is displayed in unrolled, planar form.

FIG. 8B illustrates an example of an angle θ that indicates a respective angular position that is represented by a pixel P.

FIG. 9 illustrates an example embodiment of an operational flow for reducing or eliminating artifacts in images that are generated from fluorescence-detection data.

FIG. 10 illustrates an example embodiment of an operational flow for reducing or eliminating artifacts in images that are generated from fluorescence-detection data.

FIG. 11 illustrates an example embodiment of an operational flow for detecting an artifact in OCT-detection data.

FIG. 12A illustrates an example embodiment of an OCT image.

FIG. 12B illustrates the OCT image from FIG. 12A in a different coordinate system.

FIG. 13A illustrates the respective sums of the pixel values in the OCT image that is shown in FIG. 12A.

FIG. 13B shows the differences between the respective sums of neighboring angles for the angles and sums that are shown in FIG. 13A.

FIG. 14A illustrates an example embodiment of an OCT image.

FIG. 14B illustrates the OCT image from FIG. 14A in a different coordinate system.

FIG. 15A illustrates the respective sums of the pixel values in the OCT image that is shown in FIG. 14A.

FIG. 15B shows the differences between the respective sums of neighboring angles for the angles and sums that are shown in FIG. 15A.

FIG. 16 illustrates an example embodiment of an operational flow for detecting an artifact in fluorescence-detection data.

FIG. 17 illustrates an example embodiment of an operational flow for removing an artifact from fluorescence-detection data.

FIG. 18 illustrates an example embodiment of an operational flow for replacing an artifact in fluorescence-detection data.

FIG. 19 illustrates an example embodiment of an operational flow for displaying fluorescence images in which an artifact has been replaced.

FIG. 20A illustrates fluorescence images that include an artifact.

FIG. 20B illustrates a display of fluorescence images in which the values of the pixels that correspond to the guide-wire artifact were replaced with invalid values.

FIG. 21 illustrates an example embodiment of a user interface that allows a user to select an area in which to perform artifact detection.

FIG. 22 illustrates an example embodiment of a user interface that allows a user to select an area in which to perform artifact detection.

FIG. 23 is a schematic illustration of an example embodiment of an imaging station.

FIG. 24A illustrates an example of a series of tomographic OCT images.

FIG. 24B illustrates an example a longitudinal view of a lumen that was generated from OCT-detection data.

FIG. 24C illustrates an example of a series of fluorescence images.

FIGS. 25A-B illustrate the calculation of a guide wire's angular range.

### DETAILED DESCRIPTION

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein. Furthermore, some embodiments include features from two or more of the following explanatory embodiments.

Also, as used herein, the conjunction “or” generally refers to an inclusive “or,” although “or” may refer to an exclusive “or” if expressly indicated or if the context indicates that the “or” must be an exclusive “or.” Furthermore, as used herein, the terms “first,” “second,” and so on, do not necessarily denote any ordinal, sequential, or priority relation and may be used to distinguish one member, operation, element, group, collection, set, etc. from another without expressing any ordinal, sequential, or priority relation. Thus, a first element, component, region, part, or section may be termed a second element, component, region, part, or section for purposes of distinction.

And, in the following description and in the drawings, like reference numbers designate identical, similar, or corresponding features. Also, an alphabetic suffix on a reference number may be used to indicate a specific instance of the feature identified by the reference number.

Additionally, some embodiments are set forth in the following paragraphs:

(1) A medical-imaging method comprising obtaining imaging data, wherein the imaging data include at least fluorescence-imaging data; detecting artifact data in the fluorescence-imaging data; generating modified fluorescence-imaging data by removing the artifact data or by changing the artifact data in the fluorescence-imaging data to a specified value; and generating an image based on the modified fluorescence-imaging data.

(2) The medical-imaging method of (1), wherein the imaging data further include optical-coherence-tomography-imaging (OCT-imaging) data, and wherein the artifact data is detected based on the OCT-imaging data.

(3) The medical-imaging method of (2), wherein detecting the artifact data includes detecting a first angular position that includes respective OCT-imaging values that change more than a first threshold; detecting a second angular position that includes respective OCT-imaging values that change more than a second threshold; and defining the detected range between first angular position and second angular position as the artifact data.

(4) The medical-imaging method of (1), wherein the artifact data is detected based on information of a structure that is depicted in the fluorescence-imaging data.

(5) The medical-imaging method of (4), wherein the structure is a guide wire or a guide catheter.

(6) The medical-imaging method of (1), wherein generating the modified fluorescence-imaging further includes changing the fluorescence-imaging data that are within a predetermined neighboring range of the artifact data to the specified value.

(7) The medical-imaging method of (1), wherein the specified value is zero or null.

(8) The medical-imaging method of (1), wherein regions of the image that correspond to the artifact data are shown in a predetermined color.

(9) The medical-imaging method of (1), further comprising performing an analysis of the modified fluorescence-imaging data.

(10) The medical-imaging method of (1), further comprising: (i) obtaining a selection of a distal frame of fluorescence-imaging data and specifying frames of the fluorescence-imaging data that have frame numbers that are less than a frame number of the distal frame as being artifact data; or obtaining a selection of a proximal frame of fluorescence-imaging data and specifying frames of the fluorescence-imaging data that have frame numbers that are greater than a frame number of the proximal frame as being artifact data.

(11) A medical-imaging device comprising at least one processor and at least one computer-readable storage media that is in communication with the at least one processor. The at least one computer-readable storage media stores instructions for causing the at least one processor and the at least one computer-readable storage media to obtain imaging data, wherein the imaging data include at least fluorescence-imaging data; detect artifact data in the fluorescence-imaging data; generate modified fluorescence-imaging data by removing the artifact data or by changing the artifact data in the fluorescence-imaging data to a specified value; and generate an image based on the modified fluorescence-imaging data.

(12) The medical-imaging device of (11), wherein the imaging data further include optical-coherence-tomography-imaging (OCT-imaging) data, and wherein the artifact data is detected based on the OCT-imaging data.

(13) The medical-imaging device of (12), wherein, to detect the artifact data, the instructions further cause the at least one processor and the at least one computer-readable storage media to detect a first angular position that includes respective OCT-imaging values that change more than a first threshold; detect a second angular position that includes respective OCT-imaging values that change more than a second threshold; and define the detected range between first angular position and second angular position as the artifact data.

(14) The medical-imaging device of (11), wherein the instructions further cause the at least one processor and the at least one computer-readable storage media to detect the artifact data based on information of a structure that is depicted in the fluorescence-imaging data.

(15) The medical-imaging device of (11), wherein the at least one computer-readable storage media further stores instructions for causing the at least one processor and the at least one computer-readable storage media to change the fluorescence-imaging data that are within a predetermined neighboring range of the artifact data to the specified value.

(16) The medical-imaging device of (11), wherein the specified value is zero or null.

(17) The medical-imaging device of (11), wherein regions of the image that correspond to the artifact data are shown in a predetermined color.

(18) The medical-imaging device of (11), wherein the at least one computer-readable storage media further stores instructions for causing the at least one processor and the at least one computer-readable storage media to perform an analysis of the modified fluorescence-imaging data.

(19) A medical-imaging system comprising a probe-interface subsystem; a patient-interface unit; a bendable optical-imaging device; at least one processor; and at least one computer-readable storage media that is in communication with the at least one processor. The at least one computer-readable storage media stores instructions for causing the at least one processor and the at least one computer-readable storage media to obtain imaging data, wherein the imaging data include at least fluorescence-imaging data; detect artifact data in the fluorescence-imaging data; generate modified fluorescence-imaging data by removing the artifact data or by changing the artifact data in the fluorescence-imaging data to a specified value; and generate an image based on the modified fluorescence-imaging data.

(20) The medical-imaging system of (19), wherein the probe-interface subsystem, the patient-interface unit, and the bendable optical-imaging device are configured to perform both optical-coherence-tomography imaging and fluorescence imaging.

The present disclosure generally concerns medical devices, and it describes example embodiments of an optical probe. The embodiments of the optical probe and portions thereof are described in terms of their state in a three-dimensional space. As used herein, the term “position” refers to the location of an object or a portion of an object in a three-dimensional space (e.g., three degrees of translational freedom along Cartesian x, y, and z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to six total degrees of freedom); and the term “shape” refers to a set of posture, positions, or orientations measured along the elongated body of the object.

As it is known in the field of medical devices, the terms “proximal” and “distal” are used with reference to the manipulation of an end of an instrument extending from the user to a surgical or diagnostic site. In this regard, the term “proximal” refers to the portion (e.g., a handle) of the instrument closer to the user, and the term “distal” refers to the portion (tip) of the instrument further away from the user and closer to a surgical or diagnostic site. It will be further appreciated that, for convenience and clarity, spatial terms such as “vertical,” “horizontal,” “up,” and “down” may be used herein with respect to the drawings. However, surgical instruments are used in many orientations and positions, and these terms are not intended to be either limiting or absolute.

According to some embodiments, a multimodality OCT-NIRAF imaging system, including an imaging station and a multimodality bendable optical-imaging device, is configured to acquire co-registered OCT-NIRAF images from a lumen sample, such as a cardiovascular vessel. In some embodiments, OCT images can be acquired with a swept source laser with a center wavelength of 1310 nanometers (nm) and a bandwidth of about 130 nm. NIRAF images can be acquired by exciting the lumen sample at 633 nm and detecting fluorescence emission in a wavelength range between 680 nm and 900 nm. OCT-irradiation light (OCT light) and excitation light (e.g., NIRAF-excitation light) are delivered to the sample via a single double-clad fiber (DCF) arranged inside the bendable optical-imaging device. The bendable optical-imaging device is connected to the imaging station through a patient interface unit (PIU), which provides a beam combiner (to direct the light) and a pullback unit that effectuates mechanical helical scanning. Some embodiments acquire synchronized and co-registered OCT and NIRAF data at a rate of at least 200 frames per second (fps) with a pullback speed of 10-40 mm/s, and each OCT-NIRAF image frame contains about 500 A-lines.

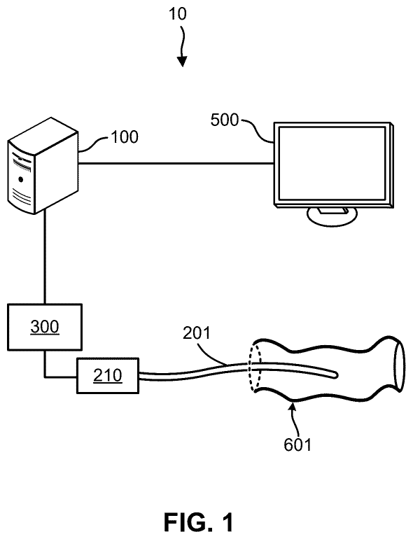

FIG. 1 is a schematic of an example embodiment of a medical-imaging system 10. The medical-imaging system 10 includes an imaging station 100, which is specially-configured computing device (e.g., desktop, laptop, server, workstation); a bendable optical-imaging device 201 (e.g., a catheter 201); a patient-interface unit (PIU) 210; a probe-interface subsystem (which includes OCT and NIRAF modules) 300; and a display device 500.

The bendable optical-imaging device 201 is an instrument that uses light guided by an imaging core (e.g., an optical probe) to look inside a body cavity or organ. For example, the bendable optical-imaging device 201 may be an endoscope, and a medical procedure in which an endoscope is inserted through a natural opening is called an endoscopy. Specialized endoscopes are generally named for how or where the endoscope is intended to be used, such as the bronchoscope (bronchi), sigmoidoscope (rectum), cystoscope (bladder), nephroscope (kidney), laryngoscope (larynx), otoscope (ear), arthroscope (joint), laparoscope (abdomen), and gastrointestinal endoscopes.

The medical-imaging system 10 is a multi-modal optical coherence tomography (MMOCT) system (e.g., a multi-modality swept-source OCT system) that includes an interferometric OCT modality and a fluorescence modality. The medical-imaging system 10 can perform both OCT imaging and fluorescence imaging (e.g., auto-fluorescence imaging, near-infrared auto-fluorescence imaging, fluorescence-lifetime imaging) and may, for example, be used for endovascular imaging and could also be adapted with a balloon catheter for esophageal imaging or imaging of other similar bodily lumens.

The bendable optical-imaging device 201 can perform optical-scanning procedures inside a lumen (e.g., vessel, bronchus, intestine, trachea, ear canal), cavity (e.g., stomach, nasal cavity) or other structure. In FIG. 1, the bendable optical-imaging device 201 extends into a sample 601 (e.g., a lumen of a patient). During an optical-scanning procedure, the probe-interface subsystem 300 generates light (e.g., OCT light, excitation light) and supplies the generated light to the bendable optical-imaging device 201. The bendable optical-imaging device 201 carries the light (e.g., OCT light, excitation light) to a distal end, where the light is emitted. And, at the distal end, the bendable optical-imaging device 201 collects light (e.g., OCT light, fluorescence light) that is emitted, scattered, or reflected by the sample 601.

The collected light is carried from the distal end of the bendable optical-imaging device 201, through the PIU 210, to the probe-interface subsystem 300. Based on the received light, the probe-interface subsystem 300 generates detection signals, which carry detection data (e.g., a series of first detection data, such as a series of groups of first detection data), and supplies the detection signals to the imaging station 100. The detection signals may include multiple modalities of detection signals, for example OCT-detection signals and fluorescence-detection signals. Accordingly, the detection data may include, for example, OCT-detection data or fluorescence-detection data.

Based on the detection data, the imaging station 100 generates one or more optical-scanning images (e.g., a series of optical-scanning images). For example, each optical-scanning image in a series of optical-scanning images may be generated from a respective group of detection data in a series of groups of detection data. The imaging station 100 supplies the one or more optical-scanning images to the display device 500, which displays the one or more optical-scanning images. For example, the imaging station 100 may generate a user interface that includes the one or more optical-scanning images and transmit the user interface to the display device 500. Also, examples of optical-scanning images include the following: OCT images (which are images that were generated from OCT-detection data), fluorescence images (which are images that were generated from fluorescence-detection data), and multi-modal images (e.g., an OCT-fluorescence image, such as a co-registered OCT-fluorescence image). Furthermore, in some embodiments, each optical-scanning image is formed by a set of frames in which each frame is a one-dimensional array of pixels.

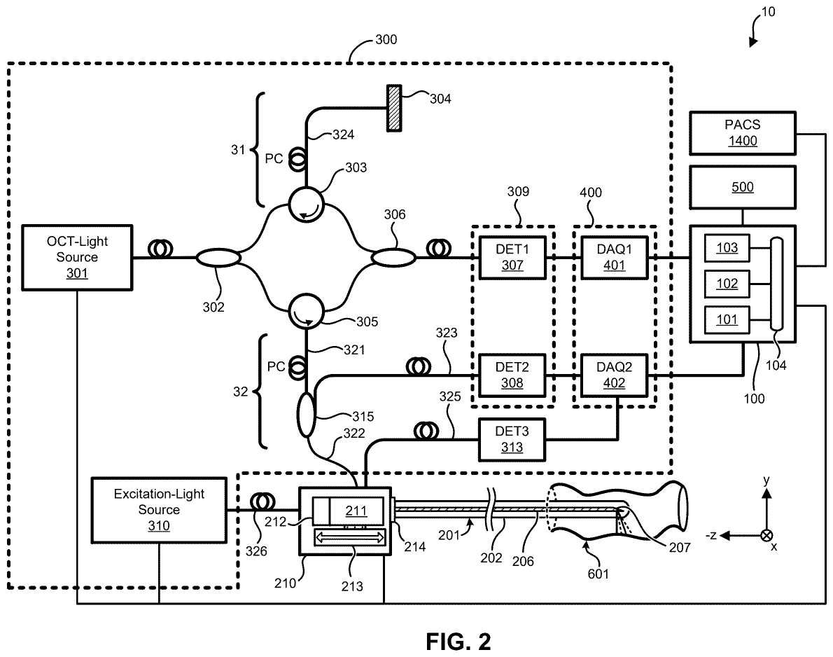

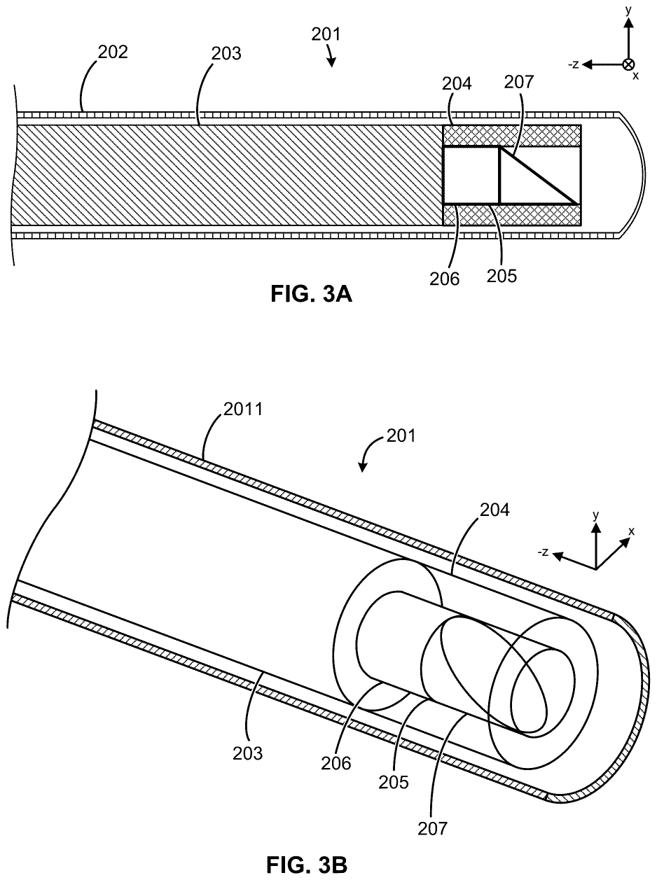

FIG. 2 is a schematic of an example embodiment of the medical-imaging system 10 of FIG. 1 that illustrates additional details of the probe-interface subsystem 300. And FIGS. 3A-B illustrate an example embodiment of a bendable optical-imaging device 201. FIG. 3A illustrates a partial sectional view of the bendable optical-imaging device 201, and FIG. 3B illustrates a partial sectional perspective view of the bendable optical-imaging device 201.

The medical-imaging system 10 includes an imaging station 100, which is specially-configured computing device (e.g., desktop, laptop, server, workstation); a bendable optical-imaging device 201; a patient-interface unit (PIU) 210; a probe-interface subsystem 300; and a display device 500. The probe-interface subsystem 300 includes an OCT-light source 301, a splitter 302, a first circulator 303, a reflector 304, a second circulator 305, a combiner 306, a first detector 307, a second detector 308, an excitation-light source 310, a third detector 313 (e.g., a photomultiplier tube (PMT), a photodetector), first data-acquisition electronics (DAQ1) 401, and second data-acquisition electronics (DAQ1) 402, as well as other members that are discussed below.

The imaging station 100 is connected to a display device 500 and an external system, such as a picture archiving and communication system (PACS) 1400.

As depicted in FIG. 2, the OCT modality is composed of an interferometer (e.g., a Michaelson interferometer) having a sample arm 32 and a reference arm 31, the OCT-light source 301, a detector unit 309, a data-acquisition unit (DAQ) 400 (which includes the first data-acquisition electronics (DAQ1) 401 and the second data-acquisition electronics (DAQ1) 402), and the imaging station 100. The sample arm 32 includes the patient interface unit (PIU) 210 and the bendable optical-imaging device 201 (e.g., a fiber-based catheter). The reference arm 31 includes a circulator 303 and a reflector 304. And the members of the OCT modality constitute an OCT module.

The fluorescence modality is composed of the excitation-light source 310, the bendable optical-imaging device 201, a third detector 313 (which may be referred to as a fluorescence detector 313), the data-acquisition unit (DAQ) 400, and the imaging station 100. In the fluorescence modality, the excitation-light source 310 is connected to the PIU 210 via an optical fiber 326. And the members of the fluorescence modality constitute a fluorescence module.

In some embodiments, a swept-source laser (1310 nm+/−50 nm) is the OCT-light source 301 for the OCT modality, and a Helium-Neon (He:Ne) laser or laser diode with a center wavelength of about 633 nm is the excitation-light source 310 for the fluorescence modality.

The bendable optical-imaging device 201 (e.g., a catheter) includes a tubular flexible body 202, a coil 203, a protector 204, and an imaging core 205 (e.g., an optical probe). The tubular flexible body 202 (e.g., a catheter sheath) surrounds the imaging core 205, and the tubular flexible body 202 may be referred to herein as a “flexible body.”

The imaging core 205 includes a double-clad optical fiber (DCF) 206 and a distal optics assembly 207. The DCF 206 may operate to carry light to the distal optics assembly 207 and to carry collected light from distal optics assembly 207 to the PIU 202. For example, a DCF 206 may transmit OCT light and collect OCT light that is reflected by a sample 601, and the DCF 206 may transmit excitation light and collect fluorescence light that is emitted by the sample 601. The distal optics assembly 207 may include a polished ball lens at the tip of the DCF 206 for side-view imaging. And the distal optics assembly 207 may include a graded index (GRIN) lens and a refractive element (grating) attached at the tip of the DCF 206. The distal optics assembly 207 may shape one or more beams of light (e.g., a beam of OCT light, a beam of excitation light), direct illuminating light to the sample 601, and collect light that is reflected from, scattered by, or emitted by the sample 601. The distal optics assembly 207 may also include a mirror that deflects one or more beams of light radially outward.

At the proximal end, the bendable optical-imaging device 201 is connected to the PIU 210 via a connector 214. The PIU 210 can spin the coil 203, for example during a pullback procedure. The coil 203 delivers torque from its proximal end to its distal end. In some embodiments, the coil 203 is fixed with, or to, the imaging core 205 such that a distal end of the imaging core 205 and the distal optics assembly 207 also spin with the coil 203, which provides the distal optics assembly 207 with a panoramic or multidirectional view. Thus, as one or more beams of light travel through the distal optics assembly 207, the distal optics assembly 207 can be rotated, thereby providing the distal optics assembly 207 with multi-directional views of the sample 601. Furthermore, the imaging core 205 (as well as the rest of the bendable optical-imaging device 201) can be translated longitudinally during the rotation, which produces a helical scanning pattern. This translation is commonly performed by pulling the distal end of the imaging core 205 back towards the proximal end, and is therefore referred to as a pullback procedure.

The PIU 210 includes a rotary junction 211 (e.g., a fiber-optic rotary joint (FORJ)), a beam combiner 212, and a pullback unit 213 (e.g., a precision linear stage), and the PIU 210 may also include the connector 214. During an optical-scanning procedure, the position of the imaging core 205, as well as the rest of the bendable optical-imaging device 201, can be adjusted or controlled by the pullback unit 213. Some embodiments of the pullback unit 213 include a rotational motor and a translation motorized stage. In some embodiments, the rotary junction 211 is located in the pullback unit 213. The rotary junction 211 allows the imaging core 205 to rotate relative to the PIU 210, for example to rotate inside the bendable optical-imaging device 201 or to rotate as the entire bendable optical-imaging device 201 rotates. During the rotation, which may be performed by the rotational motor, the imaging core 205 (as well as the rest of the bendable optical-imaging device 201) can be moved longitudinally (e.g., by a translation motorized stage) so that light (e.g., OCT light, fluorescence light) is collected in a helical scanning pattern. For example, the rotation and translation movements can helically scan the imaging core 205 inside a lumen and can produce a series of adjacent helical A-scans of the lumen, which can then be used to create a helical two-dimensional (2D) tomogram. Also for example, moving the imaging core 205 longitudinally within the lumen allows the collection of a series of B-scans, which can be combined to form a three-dimensional (3D) image of the lumen.

The imaging system 10 is configured to simultaneously acquire OCT and fluorescence images from the sample 601, which may include a biological lumen, such as a vessel. To that end, OCT light from the OCT-light source 301 is guided through the sample arm 32 to the sample 601 and through the reference arm 31 to a reflector 304 (e.g., a mirror), and the light travels back along the respective optical paths to thereby generate OCT interference patterns. Light from the light source 301 is split (e.g., 50/50) by the splitter 302 (fiber splitter or beam splitter) into a sample beam and a reference beam, which are respectively conveyed to the sample arm 32 and the reference arm 31 via respective optical fibers. In the sample arm 32, the sample beam enters the circulator 305, travels to a fiber coupler 315 via a single-mode (SM) optical fiber 321, and is delivered to the PIU 210 via a double-clad optical fiber 322. The bendable optical-imaging device 201 is connected to the PIU 210, and the PIU 210 is connected to the imaging station 100. Under control of the imaging station 100, the PIU 210 controls rotation of the imaging core 205 of the bendable optical-imaging device 201 to irradiate the sample 601 with the sample beam in a scanning manner. Light of the sample beam is reflected or scattered by the sample 601, and some of the reflected or scattered light is collected by the distal optics assembly 207 arranged at the distal end of the bendable optical-imaging device 201, and the collected light is transmitted back through the double-clad optical fiber 206 to the PIU 210. From the PIU 210, the collected light (sample beam) advances to the fiber coupler 315 through the double-clad optical fiber 322. The fiber coupler 315 conveys part of the returned light to the circulator 305 via the SM optical fiber 321, and the circulator 305 guides that part of the returned light to a combiner 306. In addition, the fiber coupler 315 forwards another part of the returned light to a second detector 308 via a multi-mode optical fiber 323.

In the reference arm 31, light of the reference beam enters a circulator 303 and is delivered to the reflector 304 via an optical fiber 324. In Time Domain OCT (TD-OCT) imaging, the reflector 304 may be implemented by a scanning mirror and an optical delay line (ODL). In a case of Frequency Domain OCT (FD-OCT) imaging, the reflector 304 may be implemented as a stationary mirror. Light of the reference beam reflected from the reflector 304 passes through the circulator 303, and is also guided to the combiner 306. In the combiner 306, the light from the reference arm 31 and the collected light from the sample arm 32 are combined. In this manner, the sample and reference beams are combined at the beam combiner 306, and then the combined sample and reference beams are supplied to a first detector 307 (which may be referred to as an OCT detector 307), which detects the combined sample and reference beams and generates interference signals according to known OCT principles.

The first detector 307 may, for example, be implemented as an array of photodiodes, a photo multiplier tube (PMT), a multi-array of cameras, or another similar interference-pattern-detecting device. In some embodiments, the first detector 307 is a balanced photodetector. The OCT-detection signals output from the first detector 307 are pre-processed (digitized) by first data-acquisition electronics (DAQ1) 401 (e.g., one or more data-acquisition circuits), and transferred to the imaging station 100. The imaging station 100 performs signal processing on the OCT-detection signals to generate OCT images in a known manner. Polarization-sensitive OCT measurements can be taken by using polarization maintaining (PM) optical fibers or through in-line paddle-based polarization controllers (PC).

A second detector 308 detects part of the sample beam transmitted from the fiber coupler 315 via a multi-mode optical fiber 323. The second detector 308 outputs an analog signal corresponding to an intensity of the backscattered light (backscattered signal). The backscattered signal returned from the sample 601 and detected by the second detector 308 is not an interference signal. The signal output from the second detector 308 is converted to digital data by the second data-acquisition electronics (DAQ2) 402. The digital signal corresponding to the intensity of the backscattered light can be used to calculate a distance or an angle at which the light from the bendable optical-imaging device 201 is incident on the sample 601. The intensity of the backscattered light may also be used as a trigger signal for starting or ending pullback and image-recording operations. Therefore, the signal output from the second detector 308 and converted to digital data by the second data-acquisition electronics (DAQ2) 402 can be used directly as a trigger signal or it can be transferred to the imaging station 100 for control processing.

The excitation-light source 310 generates and emits an excitation light (e.g., a beam of excitation light). In some embodiments, in the fluorescence modality, the excitation-light source 310 emits an excitation light with a center wavelength of 633 nm (radiation of second wavelength). In other embodiments, the excitation light can have different center wavelength (e.g., 485 nm), depending on the desired application. The excitation light is guided by a fiber 326, the rotary junction 211, the double clad fiber 206, and the distal optics 207 to irradiate the sample 601. In response to being irradiated by the excitation light, the sample 601 emits fluorescence light (e.g., near infrared auto-fluorescence (NIRAF) light, near infrared fluorescence (NIRF) light) with a broadband wavelength (radiation of third wavelength, e.g., 633 to 800 nm) in a range higher than the wavelength of the excitation light. In some embodiments, the excitation light has one of the following wavelengths or wavelength ranges: approximately 0.633 μm, 0.633-0.90 μm, and 0.500-0.700 μm.

Fluorescence is an optical phenomenon in which the molecular absorption of energy in the form of photons triggers an immediate emission of fluorescent photons with a wavelength longer than that of the excitation light. In some embodiments, the fluorescence light generated by the sample 601 includes auto-fluorescence light, which is the endogenous fluorescence light generated without application of a dye or agent. In some embodiments, the fluorescence light generated by the sample 601 includes fluorescence light generated by exogenous fluorescence of a dye or a contrast agent added to the sample (e.g., during lumen clearance). The auto-fluorescence light or the fluorescence light is collected by the distal optics 207 of the bendable optical-imaging device 201 and delivered back to the PIU 210, where the rotary junction 211 and the beam combiner 204 conveys the collected fluorescence light to the third detector 313 via an optical fiber 325. The fluorescence-detection signal (fluorescence intensity signal) output from the third detector 313 is digitized by the second data-acquisition electronics (DAQ2) 402 and transmitted to imaging station 100 for image processing. In some embodiments, the OCT-detection signal (which carries OCT-interference patterns) of the OCT modality and the fluorescence-detection signal of the fluorescence modality are simultaneously delivered to the imaging station 100.

As shown in FIG. 1, the imaging station 100 includes one or more processors 101, one or more I/O components 102, one or more computer-readable storage media 103, and one or more buses 104. The various components of the imaging station 100 are operatively connected to and communicate with each other via physical and logical data lines that are provided by the one or more buses 104. Examples of buses 104 include a universal serial bus (USB), an IEEE 1394 bus, a PCI bus, an Accelerated Graphics Port (AGP) bus, a Serial AT Attachment (SATA) bus, and a Small Computer System Interface (SCSI) bus.

The one or more processors 101 are or include one or more of the following: one or more central processing units (CPUs), such as microprocessors (e.g., a single core microprocessor, a multi-core microprocessor); one or more graphics processing units (GPUs); one or more application-specific integrated circuits (ASICs); one or more field-programmable-gate arrays (FPGAs); one or more digital signal processors (DSPs); or other electronic circuitry (e.g., other integrated circuits). Furthermore, a processor 101 may be a purpose-built controller or may be a general-purpose controller. And the one or more processors 101 are an example of a processing unit. The one or more processors 101 may operate based on computer-readable instructions (e.g., in one or more programs) stored on the one or more computer-readable storage media 103.

As used herein, a computer-readable storage medium 103 includes an article of manufacture, for example a magnetic disk (e.g., a floppy disk, a hard disk), an optical disc (e.g., a CD, a DVD, a Blu-ray), a magneto-optical disk, magnetic tape, and semiconductor memory (e.g., a non-volatile memory card, flash memory, a solid-state drive, SRAM, DRAM, EPROM, EEPROM). Computer-readable storage media 103 may be volatile memory, non-volatile memory, ROM, and RAM. And examples of the one or more computer-readable storage media 103 include networked-attached storage (NAS) devices, intranet-connected storage devices, and internet-connected storage devices. The computer-readable storage media 103 can store computer-readable data or computer-executable instructions, for example Operating System (OS) programs, control-program code, and processing-program code. Furthermore, in embodiments where the one or more computer-readable storage media 103 include RAM, the one or more processors 101 can use the RAM as a work area. Additionally, when the imaging station 100 or the one or more processors 101 are described as obtaining information or data, recording information or data, generating information or data, storing information or data, operating on information or data, processing information or data, etc., the information or data are stored in the one or more computer-readable storage media 103. Also, the one or more computer-readable storage media 103 are an example of a storage unit. And the computer-readable storage media 103 may be distributed among multiple processors 101.

The I/O components 102 include physical interfaces and communication components (e.g., a GPU, a network-interface controller) that enable communication (wired or wireless) with other members of the medical-imaging system 100 (e.g., the PIU 210, the OCT-light source 301, the first detector 307, the second detector 308, the third detector 313, the first data-acquisition electronics (DAQ1) 401, the second data-acquisition electronics (DAQ2) 402), with other computing devices (e.g., a networked computer, the PACS 1400), and with input or output devices, which may include the display device 500, a network device, a keyboard, a mouse, a printing device, a light pen, an optical-storage device, a scanner, a microphone, a drive, a joystick, and a control pad. The I/O components 102 may include programmable logic for use with a programmable logic device (PDL), such as a Field Programmable Gate Array (FPGA) or other PLD, discrete components, integrated circuitry (e.g., an Application Specific Integrated Circuit (ASIC)), or any other components including any combination thereof. The function of the I/O components 102 may be realized at least in part by computer-executable instructions (e.g., one or more programs) recorded in the one or more computer-readable storage media 103 and executed by the one or more processors 101.

The functional operation of the imaging system 10 illustrated in FIG. 2 is implemented by the one or more processors 101 of the imaging station 100 executing computer-executable instructions (e.g., one or more programs) stored in the one or more computer-readable storage media 103.

Also, in some embodiments, the imaging station 100 includes the first data-acquisition electronics (DAQ1) 401 or the second data-acquisition electronics (DAQ2) 402.

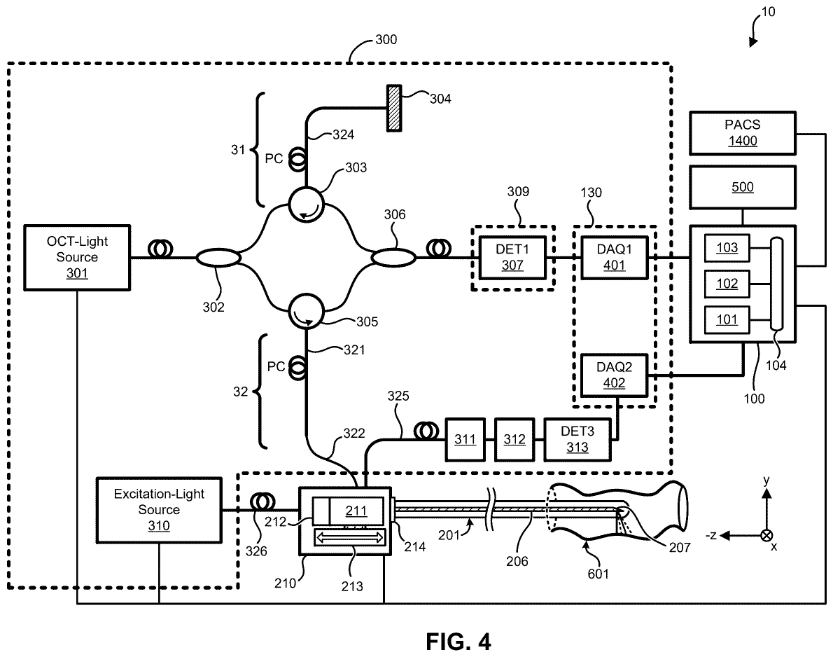

FIG. 4 is a schematic of an example embodiment of the medical-imaging system 10 of FIG. 1 that illustrates additional details of the probe-interface subsystem 300. The embodiment of the medical-imaging system 10 in FIG. 4 is similar to the embodiment of the medical-imaging system 10 in FIG. 2. However, probe-interface subsystem 300 in FIG. 4 omits the second detector 308 and the fiber coupler 315, and the probe-interface subsystem 300 in FIG. 4 includes a dichroic filter 311 and a line filter 312. The dichroic filter 311 directs the collected fluorescence light to the third detector 313. The line filter 312 reduces signal washout from any remaining back-reflected excitation light that reaches the line filter 312. For example, the line filter 312 can be narrow with a high filtering capability for the NIRAF excitation wavelength (e.g., 635 nm), with only a couple of nanometers of bandwidth, or the bandwidth can be broader (e.g., up more than 2 nm and less than 20 nm or 40 nm) to reduce Raman signals from an optical fiber that can affect NIRAF signal-to-noise ratio. Also, some embodiments of the probe-interface subsystem 300 in FIG. 2 include the dichroic filter 311 or the line filter 312.

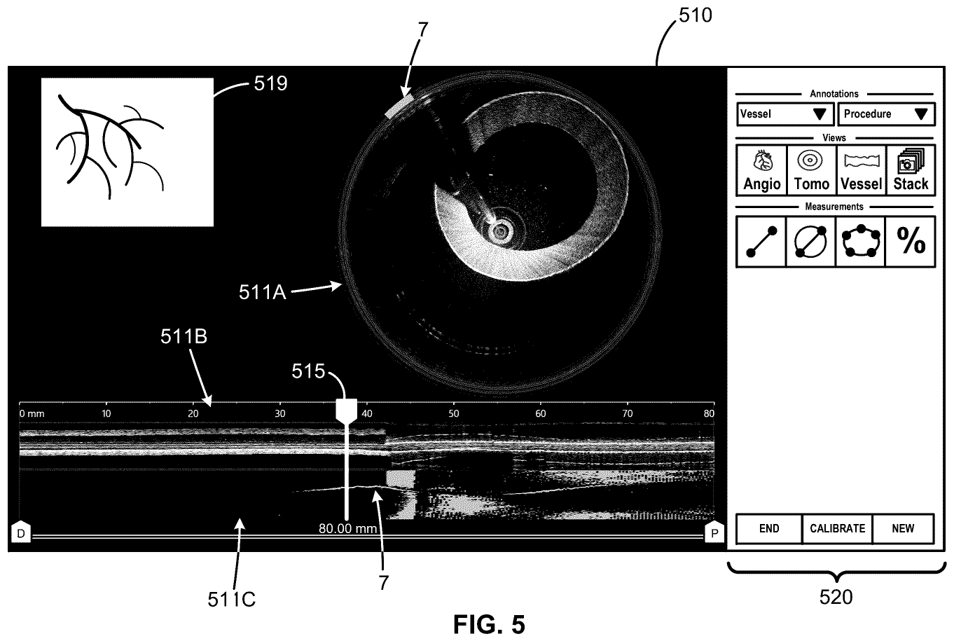

FIG. 5 illustrates an example embodiment of a screen from a user interface. The screen 510 includes three images 511A-C that were generated based on detection data. Also, the screen includes a control area 520, which includes graphical controls that, when activated, operated, or otherwise manipulated, provide inputs to the user interface. And the screen includes an angiogram image 519.

The three images 511A-C include a first image 511A, which shows a tomographic view (a lateral cross-sectional view, which is perpendicular to the axis of the vessel) of a lumen that was generated from OCT with fluorescence-detection data on the periphery; a second image 511B, which shows a longitudinal view (a longitudinal cross-sectional view, which is parallel to the axis of the vessel) of the lumen that was generated from OCT-detection data; and a third image 511C, which shows a longitudinal view of the lumen that was generated from fluorescence-detection data. The third image 511C is an unrolled view (splayed-open view) of the lumen (shows the lumen as if the lumen was unrolled to form a plane). The first image 511A is taken from a view that is orthogonal to the views from which the second image 511B and the third image 511C are taken. Additionally, a location indicator 515 indicates where the longitudinal view of the first image 511A is located in the second image 511B and in the third image 511C (e.g., where the plane that is shown in the first image 511A is located in the second image 511B and in the third image 511C).

The first image 511A and the third image 511C both show an artifact 7. In this example, the artifact 7 was caused by a guide wire. Some guide wires and some guide catheters include materials that fluoresce. Such guide wires and guide catheters will be visible in images that are generated from the detection data and will thus cause artifacts. Also, some guide wires and some guide catheters include materials that do not fluoresce. Such guide wires and guide catheters block the light (OCT light and fluorescence light) that is emitted by the bendable optical-imaging device 201 from reaching the lumen, which produces artifacts (e.g., shadows) in the images that are generated from the detection data. Not only do these artifacts affect (e.g., degrade) the view of the lumen, the artifacts also affect the analysis of the imaging data.

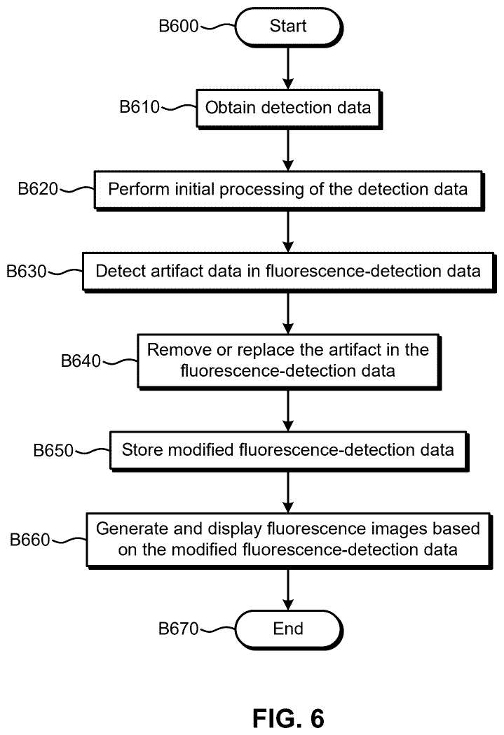

FIG. 6 illustrates an example embodiment of an operational flow for reducing or eliminating artifacts in images that are generated from fluorescence-detection data. Although this operational flow and the other operational flows that are described herein are each presented in a certain order, some embodiments may perform at least some of the operations in different orders than the presented orders. Examples of different orders include concurrent, parallel, overlapping, reordered, simultaneous, incremental, and interleaved orders. And some embodiments of the operational flows may include blocks from two or more of the operational flows that are described herein. Thus, other embodiments of the operational flows that are described herein may omit blocks, add blocks, change the order of the blocks, combine blocks, or divide blocks into more blocks.

Furthermore, although some of the operational flows that are described herein are performed by an imaging station for simplicity of description, some embodiments of these operational flows are performed by two or more imaging stations or by one or more other specially-configured computing devices.

The flow begins in block B600 and moves to block B610, where an imaging station 100 obtains (e.g., collects) detection data from a probe-interface subsystem 300. The detection data include fluorescence-detection data. In block B620, the imaging station 100 performs initial processing of the detection data. The initial processing may include generating one or more images (including fluorescence images) based on the detection data. The initial processing may also include forming a data array of a three-dimensional (3D) structure, performing FFT (fast Fourier transform), performing polar-to-cartesian-coordinate conversion, detecting a lumen border, on OCT data, and the initial processing may include forming a two-dimensional (2D) data array and distance correction (based on lumen border detection of OCT data) on the fluorescence data.

When obtaining detection data that includes both fluorescence-detection data and OCT-detection data (e.g., in block B610), an OCT module performs sweeping of the wavelength of OCT light 500 times as the imaging core 205 of the catheter 201 makes one rotation. During that one wavelength scan cycle of the OCT light source 301, the information along the beam in the depth direction is captured in the form of optical interference and is received as a high speed optical signal. The captured optical signal is converted to an electrical signal by a fast, opto-electrical detector (e.g., the first detector 307), such as a photodiode or a photomultiplier. The converted electrical signal is then further converted from an analog signal to a digital signal (e.g., by DAQ1 401). Data of each line along the beam is often called A-line data, and this data constitutes a data set along the radial line of the tomographic view. One frame consists of one rotation of the imaging core 205 and thus 500 radial lines of A-line data. One datum of fluorescence is obtained using a photomultiplier (e.g., the third detector 313) at each one of 500 positions per one rotation of the imaging core 205 of the catheter 201. The electrical signal of the photomultiplier is also converted to a digital signal by an A/D converter (e.g., by DAQ2 132). These data of OCT and fluorescence may be collected for 400 frames of tomographic view, or 400 rotations of the imaging core 205 of the catheter.

When there are 512 data in the depth direction for OCT imaging, one rotation of the imaging core 205 creates a dataset of 512 by 500. With 400 rotations for one pullback, the medical-imaging system 10 collects a OCT-detection data set of 512 by 500 by 400. In contrast, fluorescence imaging will have 500 data per one rotation of the imaging core. With 400 rotations per one pullback, the fluorescence-detection data collected is an array of 500 by 400. The tomographic image of OCT has corresponding 500 fluorescence-detection data for one tomographic image of fluorescence.

An example of initial processing of data in block B620 is performed as follows. In this example, one frame of OCT-detection data in the tomographic plane is 512 by 500. This constitutes a polar image of one frame of OCT. In that same tomographic plane, the fluorescence-detection data consists of just a one-dimensional array of 500 data. The OCT-detection-data array is processed by Fourier transform (FFT, fast-Fourier transform) to obtain the reflectance and scattering along the line of the beam. The OCT-detection data is converted from polar coordinates to cartesian coordinates in each of the frames. After the polar-to-cartesian conversion, the OCT-detection dataset is adjusted to 500 frames (500 tomographic images) of 1024 by 1024 data. The lumen-border detection is performed within the A-lines, and the distances from the center to the lumen are detected. The lumen-detection algorithms can be applied either on the polar-coordinates representation of the OCT-detection data or on the cartesian-coordinates representation of the OCT-detection data. This distance is used to correct the fluorescence signal, and the distance correction is applied to the fluorescence-detection data to calculate distance-corrected fluorescence data. In some embodiments, artifact detection is performed on the fluorescence-detection data before the distance correction is applied.

Fluorescence-detection data upon which initial processing has been performed are referred to herein as initially-processed fluorescence-detection data.

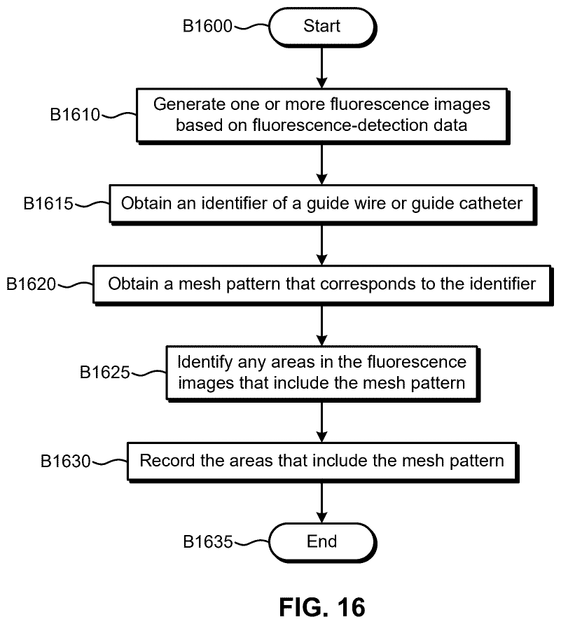

Then, in block B630, the imaging station 100 detects artifact data in the fluorescence-detection data, for example by detecting the artifact data in one or more fluorescence images (e.g., fluorescence frames). Artifact data includes detection data that was generated from the imaging of an artifact, such as a guide wire or a guide catheter. Also, in the following description, detecting artifact data may be referred to simply as detecting an artifact. Some embodiments of block B630 include the operational flow in FIG. 16. And some embodiments of the imaging station 100 use a trained machine-learning model (e.g., an artificial neural network) to detect artifact data (i.e., to detect an artifact).

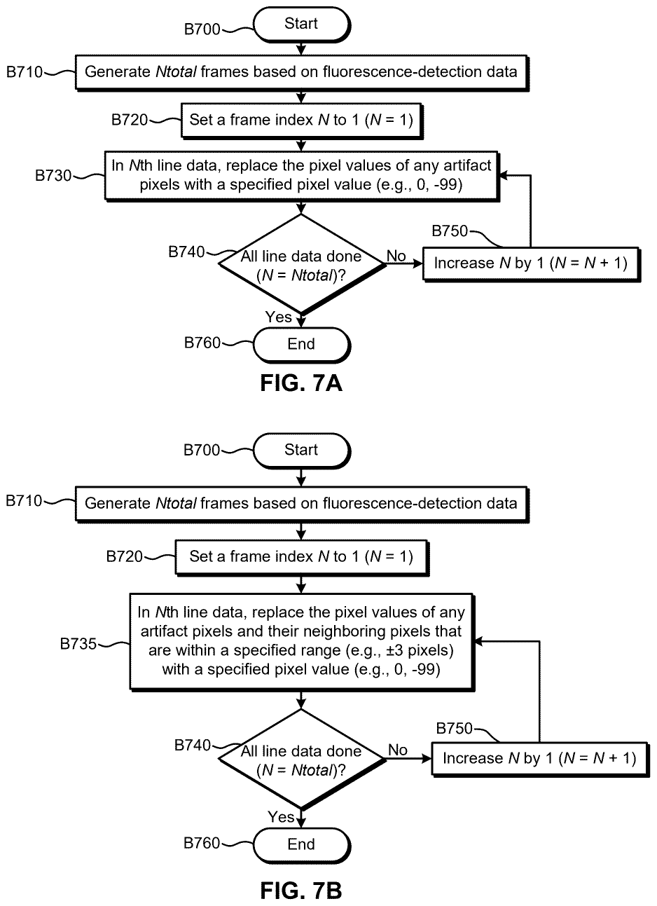

Next, in block B640, the imaging station 100 removes or replaces the artifact in the fluorescence-detection data by altering the data in the fluorescence-detection data that corresponds to the artifact. For example, the imaging station 100 may remove or replace the artifact in the fluorescence-detection data as described in FIG. 7A or 7B.

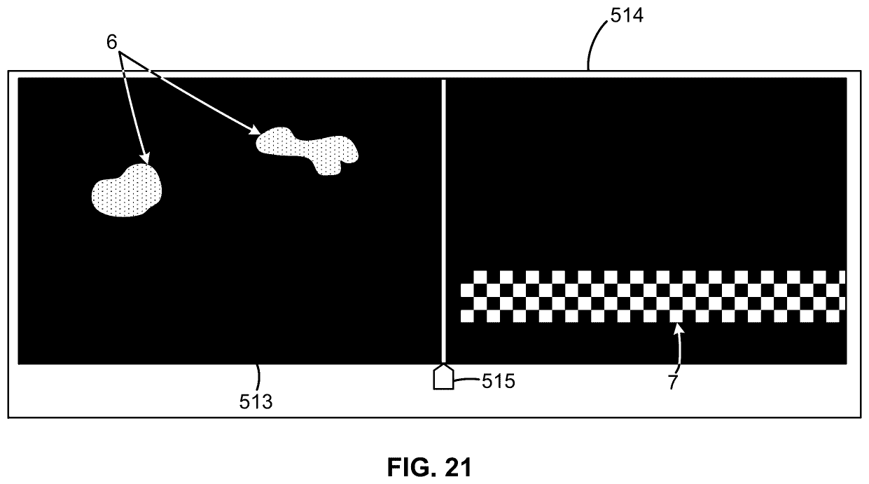

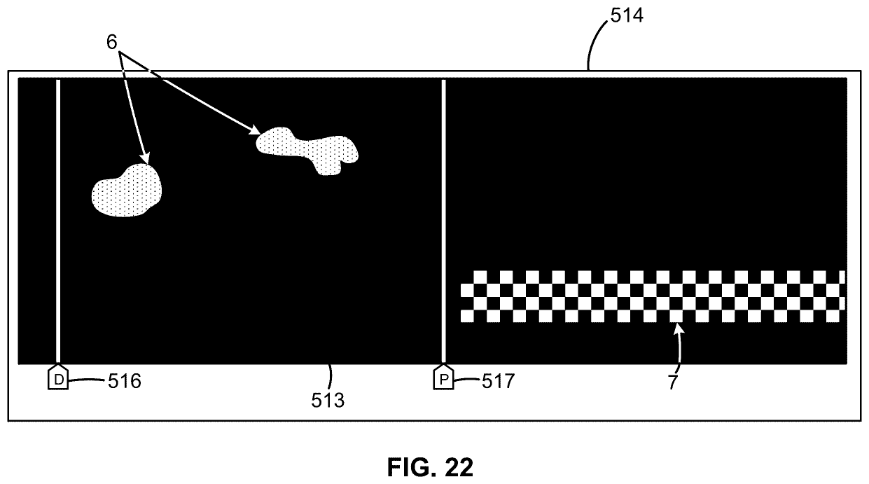

Furthermore, in some embodiments, the imaging station 100 receives an input from a user that indicates the images in which the imaging station 100 will attempt to detect artifacts in block B630 and will remove or replace detected artifacts in block B640. Example embodiments of user interfaces that can receive such inputs (the distal and proximal limits of the artifact detection) are shown in FIGS. 21 and 22. Also, some user interfaces allow a user to provide such inputs by entering one or more image numbers (frame numbers). In some embodiments, the selections of the distal and proximal limits of the artifact detection are done by the imaging station 100 using an auto-detection algorithm of the guide catheter in fluorescence images, OCT images, or both fluorescence images and OCT images. The auto-detection algorithm can be any one or combination of pattern matching, thresholding on the fluorescence image, pattern matching on the OCT image, and detection algorithms that are based on machine learning on fluorescence or OCT images. Further, the markers in FIGS. 21 and 22 can be used on a user interface to confirm or to correct and adjust the selection by the auto-detection algorithm.

In block B650, the imaging station 100 stores the modified fluorescence-detection data. Then, in block B660, the imaging station 100 generates one or more fluorescence images based on the modified fluorescence-detection data and displays the one or more images on a display device 500. Finally, the flow ends in block B670.

FIG. 7A illustrates an example embodiment of an operational flow for removing or replacing an artifact in fluorescence-detection data. Before starting the operational flow, the imaging station 100 has obtained a set of Ntotal tomographic images (frames). A frame is a tomographic image in OCT imaging. In fluorescence imaging, a frame is just one line data (in a tomographic-image plane), not 2D data. The carpet view of the fluorescence modality is the splayed-open view (the unrolled view).

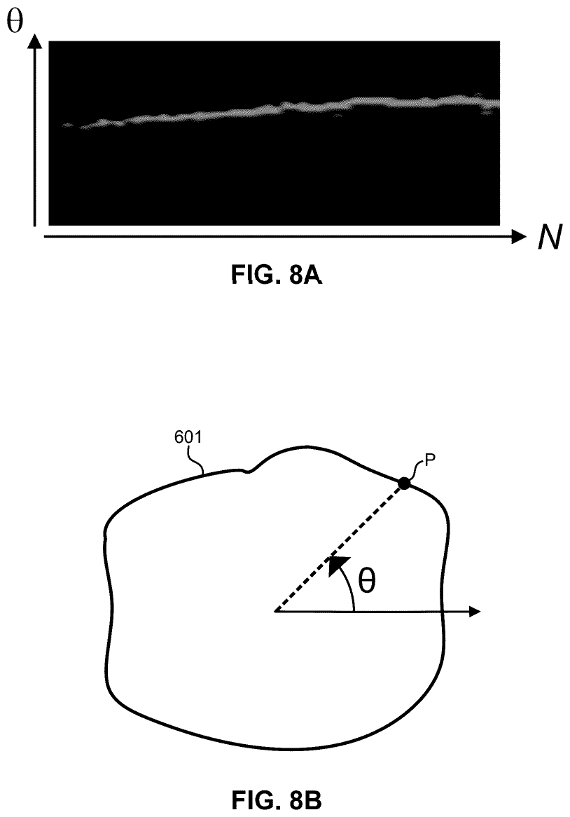

The flow starts in block B700 and then proceeds to block B710, where an imaging station generates fluorescence frames based on fluorescence-detection data. And, in block B720, the imaging station 100 sets a frame index N to 1. Each fluorescence frame may consist of a respective set of line data that is one pixel wide. For example, FIG. 8A illustrates an example embodiment of a set of fluorescence frames that is displayed in unrolled, planar form. The unrolled, planar form may be represented by a matrix A (N, θ). In FIG. 8A, the horizontal axis is the frame index, and the vertical axis is the angle θ. As shown in FIG. 8B, the angle θ indicates the respective angular position that is represented by a pixel P (e.g., the angular position of the distal optics assembly 207 at the time when the detection data that corresponds to the pixel P was captured).

Then, in block B730, the imaging station 100 replaces the respective pixels values of any artifact pixels in the Nth set of line data (the Nth line data) with a specified pixel value. For example, in embodiments in which the imaging station 100 removes the artifact, the imaging station 100 may replace the pixel values that correspond to the artifact with zero or another value that is both (1) a valid pixel value (i.e., a pixel value that is valid for display) and (2) a value that indicates that either no fluorescent light was detected or that an invalid fluorescence signal (artifact) was detected at the pixel. For example, in embodiments in which the imaging station 100 replaces the artifact, the imaging station 100 may replace the pixel values that correspond to the artifact with a value that is not a valid pixel value for display (e.g., −99, +999999, null).

Next, in block B740, the imaging station 100 determines whether block B730 has been performed for every set of line data (whether N=Ntotal). In the above explanation, Ntotal is 400, that is, 400 is the total number of rotations for one pullback, and is thus also the total number of sets of line data. If the imaging station 100 determines that block B730 has not been performed for every set of line data (B740=No), then, in block B750, the imaging station 100 increases N by 1, and the flow returns to block B730. If the imaging station 100 determines that block B730 has been performed for every set of line data (B740=Yes), then the flow ends in block B760.

FIG. 7B illustrates an example embodiment of an operational flow for removing or replacing an artifact in fluorescence-detection data. The operational flow in FIG. 7B replaces block B730 in FIG. 7A with block B735. In block B735, in the Nth set of line data, the imaging station 100 replaces the pixel values of any artifact pixels and their neighboring pixels that are within a specified range with the specified pixel value. The specified range may be defined as a pixel range (e.g., +/−3 pixels, 8 pixels, 10 pixels) or as an angular range (e.g., +/−3°, 5°, 7°, 10°, 15°). For example, in embodiments in which the imaging station 100 removes the artifact, the imaging station 100 may replace the pixel value of pixels that are within the specified range of any artifact pixel with 0 or another value that is both (1) a valid pixel value (i.e., a pixel value that is valid for display) and (2) a value that indicates that either no fluorescent light was detected or that an invalid fluorescence signal (artifact) was detected at the pixel. For example, in embodiments in which the imaging station 100 replaces the artifact, the imaging station 100 may replace the pixel value of pixels that are within the specified range of any artifact pixel with a value that is not a valid pixel value for display (e.g., −99, +999999, null). Thus, in FIG. 7B, the values of any pixels that are sufficiently close to the artifact pixels are also modified.

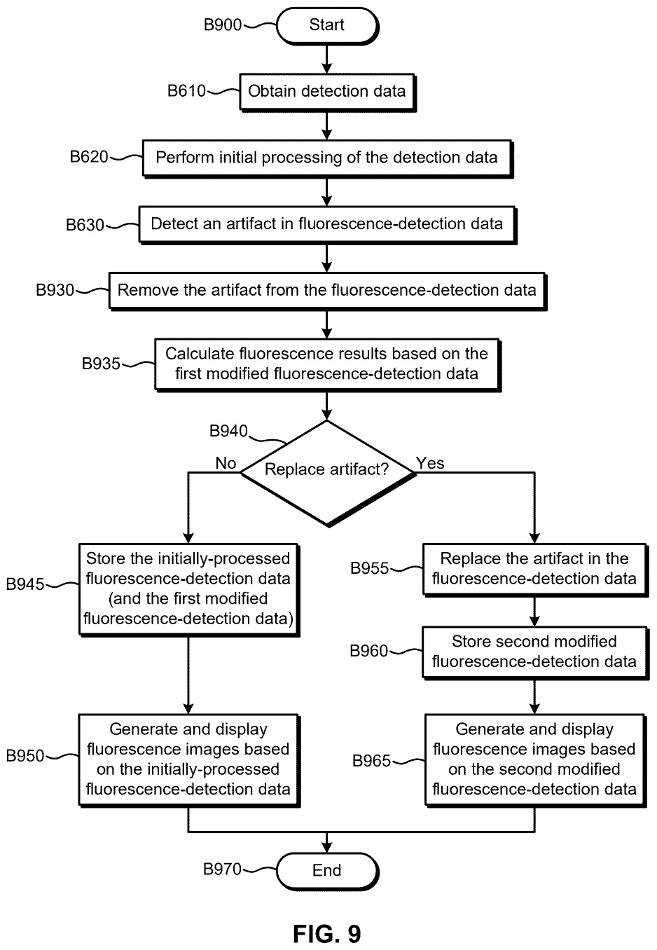

FIG. 9 illustrates an example embodiment of an operational flow for reducing or eliminating artifacts in images that are generated from fluorescence-detection data. This embodiment further calculates a statistical value, or other numerical calculations, based on the fluorescence-imaging results, after the removal of artifacts. By doing so, this embodiment eliminates the erroneous statistical analysis of the vessel, by removing the artifacts which are not representative of the vessel signals of interest. This embodiment saves and displays images with the artifact removed or replaced with specified value or this embodiment saves and displays the initial image with artifacts included as is. The flow starts in block B900 and then performs blocks B610, B620, and B630. Blocks B610, B620, and B630 in FIG. 9 are similar or identical to blocks B610, B620, and B630 in FIG. 6, and redundant descriptions thereof are omitted. In block B930, the imaging station 100 removes the artifact from the fluorescence-detection data. For example, the imaging station may remove the artifact from the fluorescence-detection data by performing the operational flow in FIG. 7A or the operational flow in FIG. 7B using a value that is both (1) a valid pixel value (i.e., a pixel value that is valid for display) and (2) a value that indicates that either no fluorescent light was detected or that an invalid fluorescence signal (artifact) was detected at the pixel as the specified pixel value in block B730. if block B730 is performed, and, if block B735 is performed, as the specified pixel value in block B735. The fluorescence-detection data from which the artifact has been removed constitutes first modified fluorescence-detection data.

Next, in block B935, the imaging station 100 calculates one or more fluorescence results based on the first modified fluorescence-detection data. Examples of fluorescence results include the following: a maximum value per image (e.g., NIRAF maximum value per image); an image sum (e.g., NIRAF image sum); a moving sum (e.g., NIRAF moving sum), such as a moving sum of image sums and a maximum moving sum of image averages; a fluorescence count (e.g., NIRAF counts); fluorescence index (e.g., NIRAF index); and maximum values of moving sums, fluorescence counts, or fluorescence indices.

In the following description, N is an image in a set of fluorescence images that can be represented by matrix A (N, θ) in unrolled, planar form

In some embodiments, the fluorescence maximum value per image (frame) MPF of image N can be described by equation (1):

$$MPF ( N ) = {max}_{i} \{ A ( N , i ) \} . \qquad ( 1 )$$

In some embodiments, the fluorescence image (frame) sum FS of image N can be described by equation (2):

$$FS ( N ) = {\sum }_{i} A ( N , i ) . \qquad ( 2 )$$

In some embodiments, the moving sum of image (frame) sums MSFS of image N and the following k images can be described by equation (3):

$$MSFS ( N ) = {\sum }_{j = N}^{N + k} {\sum }_{i} A ( N , i ) . \qquad ( 3 )$$

In some embodiments, the maximum value MaxMSFS in the set of MSFS values of Ntotal images (N=1 to N=Ntotal) can be described by equation (4):

$$Max MSFS ( N ) = max \{ {\sum }_{j = N}^{N + k} {\sum }_{i} A ( N , i ) {\vert}_{N = 1}^{N = {N}_{Total}} \} . \qquad ( 4 )$$

Next, in block B940, the imaging station 100 determines whether to replace the artifact. For example, the imaging station 100 may determine whether an instruction has been received to replace the artifact or whether a setting has been set to enable artifact replacement. If the imaging station 100 determines not to replace the artifact (B940=No), then the flow moves to block B945. In block B945, the imaging station 100 stores the initially-processed fluorescence-detection data, which was generated in block B620, and, in some embodiments, stores the first modified fluorescence-detection data. Then, in block B950, the imaging station 100 generates and displays fluorescence images based on the initially-processed fluorescence-detection data. And the flow then ends in block B970. Alternatively, not replacing the artifact (B940=No) could be processed to show the first modified fluorescence-detection data generated in B930.

If the imaging station 100 determines to replace the artifact (B940=Yes), then the flow proceeds to block B955. In block B955, the imaging station 100 replaces the artifact in the fluorescence-detection data, based on the detection results on B630. For example, the imaging station 100 may replace the artifact in the fluorescence-detection data by performing the operational flow in FIG. 7A or the operational flow in FIG. 7B using a value that is not a valid pixel value for display (e.g., −99, +999999, null) as the specified pixel value in block B735. And the replacement of the artifact can be performed by replacing the artifact in the first modified fluorescence-detection data. The fluorescence-detection data in which the artifact has been replaced constitutes second modified fluorescence-detection data.

Next, in block B960, the imaging station 100 stores the second modified fluorescence-detection data. And, in block B965, the imaging station 100 generates and displays fluorescence images based on the second modified fluorescence-detection data. Then the flow ends in block B970.

Furthermore, in some embodiments, the imaging station 100 receives an input from a user that indicates the images in which the imaging station 100 will attempt to detect artifacts in block B630, will remove detected artifacts in block B930, and will replace detected artifacts in block B955. Example embodiments of user interfaces that can receive such inputs are shown in FIGS. 21 and 22. Also, some user interfaces allow a user to provide such inputs by entering one or more image numbers (frame numbers). In some embodiments, the imaging station 100 also stores the initially-processed fluorescence-detection data in block B960 or stores the initially-processed fluorescence data in block B620 and then goes through removing the artifact in block B930, replacing the artifact in block B955, and storing the second modified fluorescence-detection data in block B960. In such embodiments, the initially-processed fluorescence-detection data with and without the artifact removed are both saved, for future reviewing, processing, or analysis of either or both of the sets of data. And, in such embodiments, a display can be toggled between showing the initially-processed fluorescence-detection data with the artifact removed and showing the initially-processed fluorescence-detection data without the artifact removed.

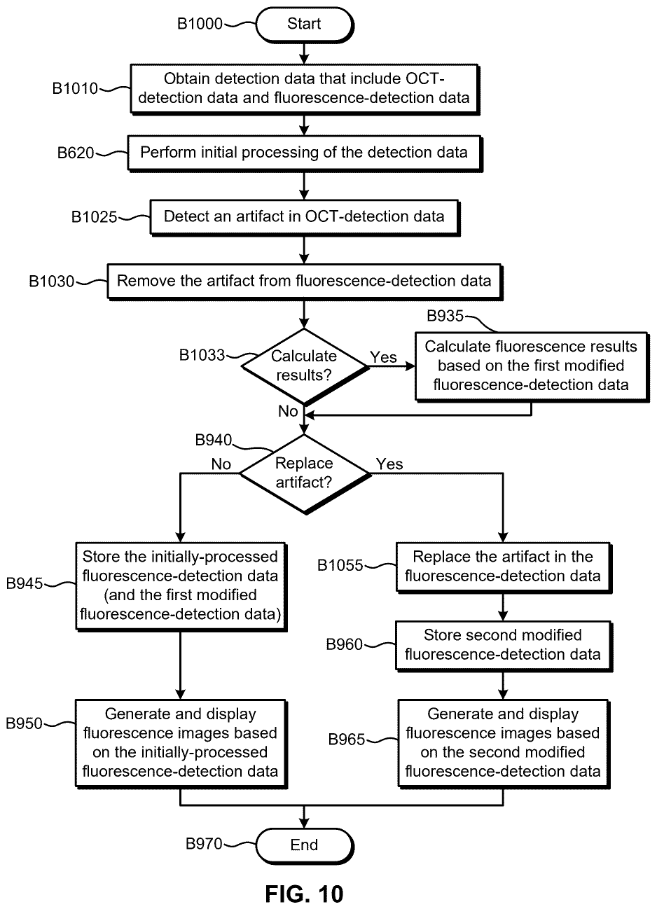

FIG. 10 illustrates an example embodiment of an operational flow for reducing or eliminating artifacts in images that are generated from fluorescence-detection data.

Block B620 in FIG. 10 is similar or identical to block B620 in FIG. 6, and a redundant description thereof is omitted. Also, blocks B935-B950 and B960-B970 in FIG. 10 are similar or identical to blocks B935-B950 and B960-B970 in FIG. 9, and redundant descriptions thereof are omitted.

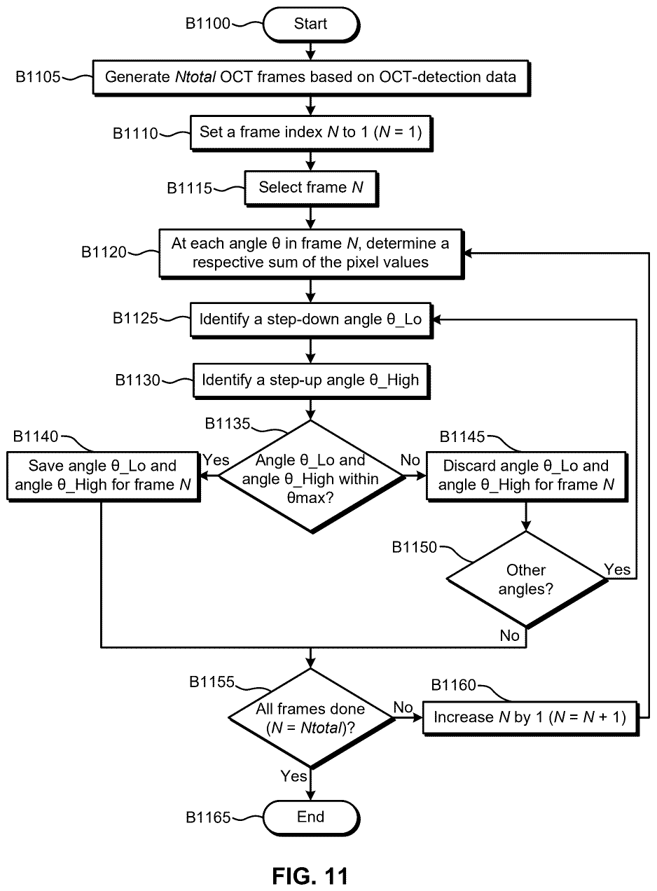

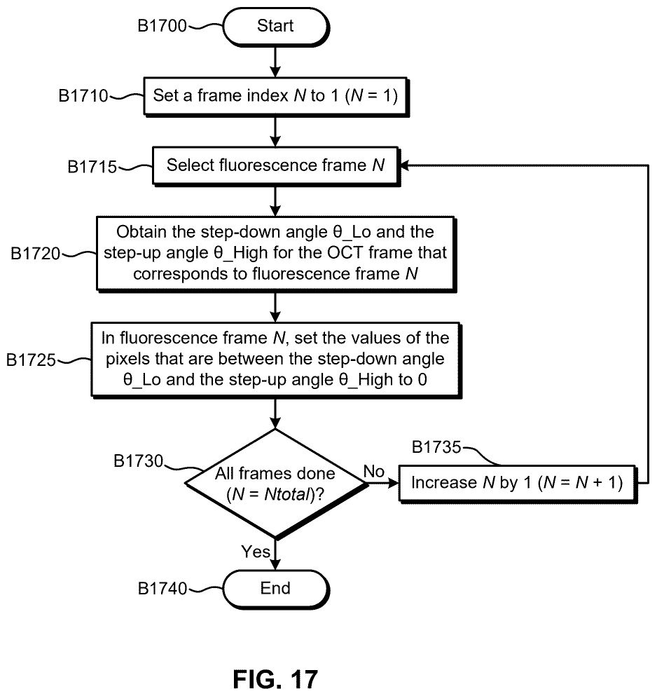

The flow starts in block B1000 and moves to block B1010, where an imaging station 100 obtains detection data that include OCT-detection data and fluorescence-detection data. The OCT-detection data and the fluorescence-detection data are co-registered. In block B1025, the imaging station 100 detects an artifact in the OCT-detection data, for example as described in FIG. 11. Then, in block B1030, the imaging station 100 removes the artifact from the fluorescence-detection data, for example as described in block B930 in FIG. 9 or in blocks B1700 to B1740 in FIG. 17.

The flow then moves to block B1033, where the imaging station 100 determines whether to calculate fluorescence results. For example, the imaging station 100 may determine whether an instruction has been received to calculate fluorescence results or whether a setting has been set to implement the calculation of fluorescence results. If the imaging station determines to calculate fluorescence results (B1033=Yes), then the flow moves to block B935 and then to block B940. If the imaging station 100 determines not to calculate fluorescence results (B1033=No), then the flow proceeds to block B940.

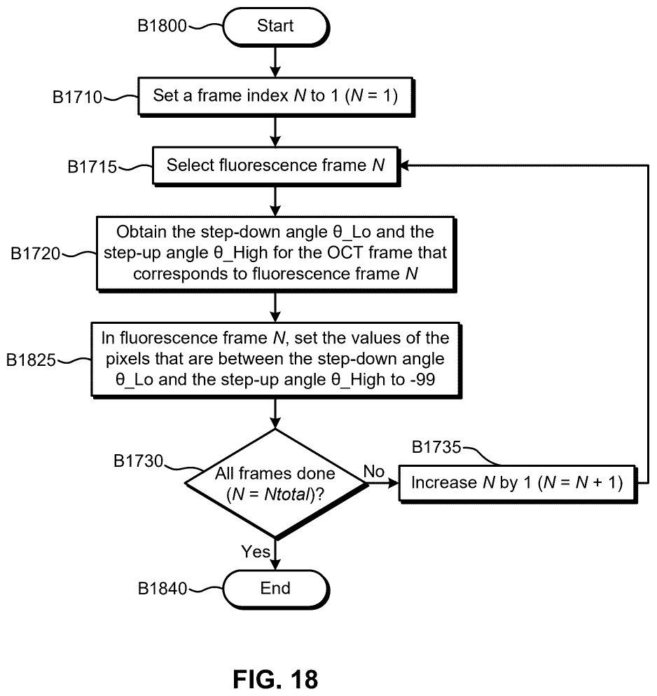

In block B1055, the imaging station replaces the artifact in the fluorescence-detection data, for example as described in block B955 in FIG. 9 or in blocks B1710 to B1840 in FIG. 18.

Furthermore, in some embodiments, the imaging station 100 receives an input from a user that indicates the images in which the imaging station 100 detects artifacts in block B1025, removes detected artifacts in block B1030, and replaces detected artifacts in block B1055. Example embodiments of user interfaces that can receive such inputs are shown in FIGS. 21 and 22. Also, some user interfaces allow a user to provide such inputs by entering one or more image numbers (frame numbers).

FIG. 11 illustrates an example embodiment of an operational flow for detecting an artifact in OCT-detection data. The flow starts in block B1100 and then moves to block B1105, where an imaging station 100 generates Ntotal (where Ntotal is a positive integer) OCT frames based on OCT-detection data. Next, in block B1110, the imaging station 100 sets a frame index N to 1. In block B1115, the imaging station 100 selects frame N.

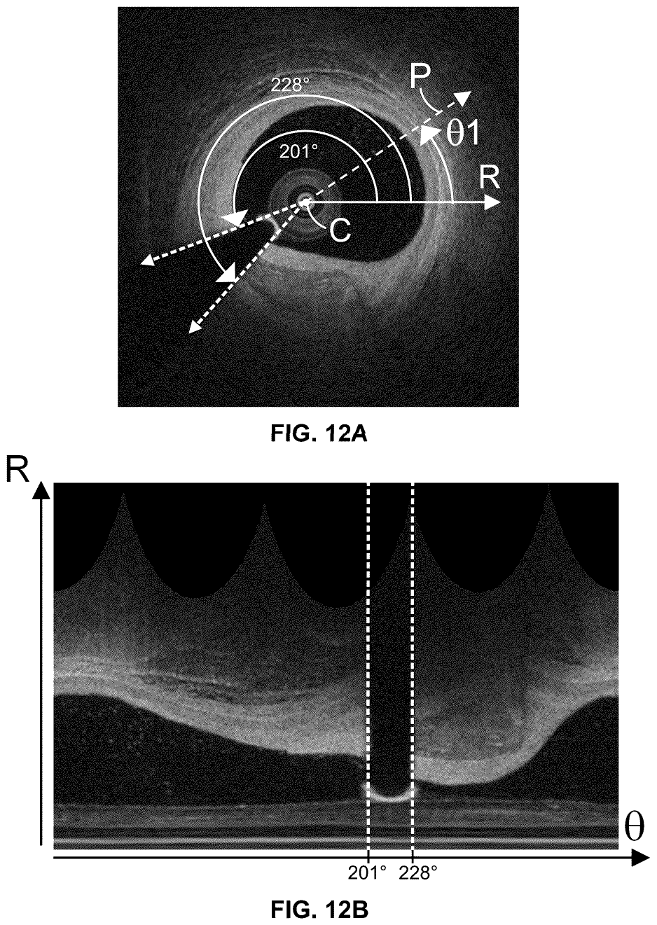

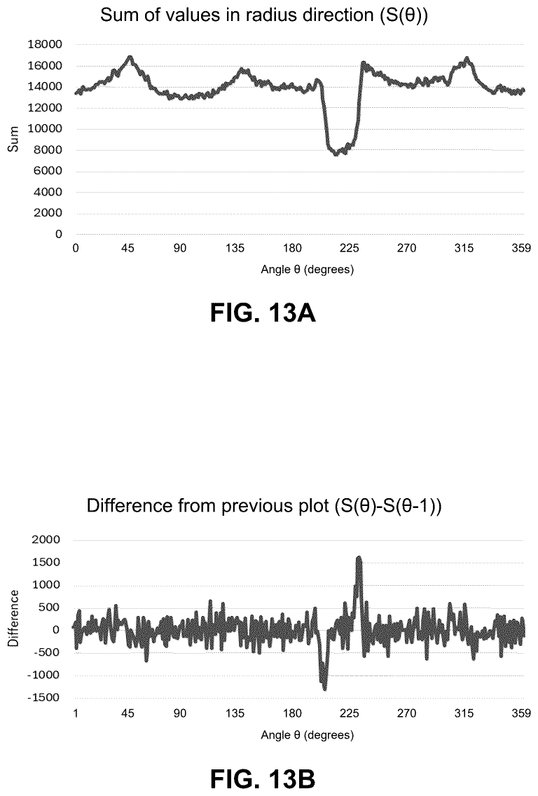

Next, in block B1120, at each angle θ in frame N, the imaging station 100 determines a respective sum of the pixel values. The respective sum of the pixel values for an angle θ is the sum of the pixel values that lie along a path in the radial direction from the center of the frame to the edge of the frame, and the angle of the path is the angle θ. For example, FIG. 12A illustrates an example embodiment of an OCT frame. Line R indicates the reference direction (e.g., polar axis) of the angles. The sum of the pixel values for angle θ1 is the sum of the pixel values of the pixels that lie along a path P in a radial direction from the center C of the frame to the edge of the frame, where the radial direction and the reference direction (indicated by line R) form angle θ1. The OCT frame may also have a different format than the format that is shown in FIG. 12A. For example, FIG. 12B illustrates the OCT frame from FIG. 12A in a different coordinate system. In FIG. 12B, the pixels are arranged such that the lower-left corner indicates an angle of 0° and a radial distance R of 0. The vertical axis indicates radial distance R, and the horizontal axis indicates angle θ. Also, FIG. 13A illustrates the respective sums of the pixel values in the OCT frame that is shown in FIG. 12A. In FIG. 13A, the horizonal axis indicates angle θ, and the vertical axis indicates S(θ), which is angle θ's respective sum.

Next, in block B1125, the imaging station 100 identifies a step-down angle θ_Lo, which is an angle where the sums have an abrupt step-down change. A step-down angle θ_Lo may indicate the following: (i) The absolute value of the difference between (1) the respective sum of angle θ_Lo and (2) the respective sum of an angle that precedes angle θ_Lo and that is within a specified range (e.g., 1°, 3°, 5°, 8°) of angle θ_Lo is greater than a step-down threshold, and the respective sum of the angle that precedes angle θ_Lo is greater than the respective sum of angle θ_Lo (and thus indicates a step down in value from the angle that precedes angle θ_Lo to angle θ_Lo). (ii) The absolute value of the difference between (1) the respective sum of angle θ_Lo and (2) the respective sum of an angle that follows angle θ_Lo and that is within a specified range (e.g., 1°, 3°, 5°, 8°) of angle θ_Lo is greater than a step-down threshold, and the respective sum of the angle that follows angle θ_Lo is less than the respective sum of angle θ_Lo (and thus indicates a step down in value from angle θ_Lo to the angle that follows angle θ_Lo).

For example, FIG. 13B shows the differences between the respective sums of neighboring angles for the angles and sums that are shown in FIG. 13A. In FIG. 13B, the vertical axis indicates the difference between each angle's sum and the sum of the previous angle. Thus, for angle θ, the vertical axis shows S(θ)-S(θ-1) (which are shown in FIG. 13A). In FIG. 13B, 201° is a step-down angle θ_Lo (an angle that has an abrupt step-down change), and the graph in FIG. 13B shows a peak at 201°. Also, 201° is marked in FIGS. 12A and 12B.

Next, in block B1130, the imaging station 100 identifies a step-up angle θ_High, which is an angle where the sums have an abrupt step-up change. A step-up angle θ_High may indicate the following: (i) The absolute value of the difference between (1) the respective sum of angle θ_High and (2) the respective sum of an angle that precedes angle θ_High and that is within a specified range (e.g., 1°, 3°, 5°, 8°) of angle θ_High is greater than a step-up threshold, and the respective sum of the angle that precedes angle θ_High is less than the respective sum of angle θ_High (and thus indicates a step up in value from the angle that precedes angle θ_High to angle θ_High). (ii) The absolute value of the difference between (1) the respective sum of angle θ_High and (2) the respective sum of an angle that follows angle θ_High and that is within a specified range (e.g., 1°, 3°, 5°, 8°) of angle θ_High is greater than a step-up threshold, and the respective sum of the angle that follows angle θ_High is greater than the respective sum of angle θ_High (and thus indicates a step up in value from angle θ_High to the angle that follows angle θ_High).

For example, in FIGS. 13A and 13B, 228° is a step-up angle θ_High (an angle that has an abrupt step-up change), and the graph in FIG. 13B shows a peak at 228°. Also, 228° is marked in FIGS. 12A and 12B. An artifact is visible from 201° to 228°.

Also, if there is more than one step-down angle θ_Lo or more than one step-up angle θ_High, the step-down angle θ_Lo or the step-up angle θ_High may respectively be the step-down angle θ_Lo or the step-up angle θ_High that is closest to the angle that has the highest respective sum.

Next, in block B1135, the imaging station 100 determines whether the step-down angle θ_Lo and the step-up angle θ_High are within a specified angular range θmax (whether |θ_High-θ_Lo|≤θmax). For example, in some embodiments θmax is 10°, 20°, 30°, or 40°. If the step-down angle θ_Lo and the step-up angle θ_High are within the specified angular range θmax (B1135=Yes), then the flow moves to block B1140, where the imaging station 10 saves the step-down angle θ_Lo and the step-up angle θ_High for frame N in association with frame N. The flow then proceeds to block B1155.

If the step-down angle θ_Lo and the step-up angle θ_High are not within the specified angular range θmax (B1135=No), then the flow moves to block B1145, where the imaging station 10 discards the step-down angle θ_Lo and the step-up angle θ_High for frame N. The flow then proceeds to block B1150. Also, in some embodiments, if the step-down angle θ_Lo and the step-up angle θ_High are not within the specified angular range θmax, the imaging station 100 increases the specified angular range θmax and then determines whether the step-down angle θ_Lo and the step-up angle θ_High are within the increased angular range θmax. If the step-down angle θ_Lo and the step-up angle θ_High are within the increased angular range θmax, then the flow moves to block B1140. If the step-down angle θ_Lo and the step-up angle θ_High are not within the increased angular range θmax, then the flow moves to block B1145.

In block B1150, the imaging station 100 determines whether to perform block B1130 for a different step-down angle θ_Lo or a different step-up angle θ_High. In some embodiments, the imaging station 100 determines to perform block B1135 again if block B1135 has not been performed for every possible combination of the step-down angles θ_Lo and the step-up angles θ_High in frame N. For example, if frame N includes two step-down angles θ_Lo and two step-up angles θ_High, then the imaging station 100 may perform block B1135 four times, each time with a different combination of the step-down angles θ_Lo and the step-up angles θ_High. If the imaging station 100 determines to perform block B1135 for a different step-down angle θ_Lo or a different step-up angle θ_High (B1145=Yes), then the flow returns to block B1125. If the imaging station 100 determines not to perform block B1135 for a different step-down angle θ_Lo or a different step-up angle θ_High (B1150=No), then the flow moves to block B1155.

In block B1155, the imaging station 100 determines whether blocks B1120-B1150 have been performed for every frame (whether N=Ntotal). If the imaging station 100 determines that blocks B1120-B1150 have not been performed for every frame (N<Ntotal) (B1155=No), then the flow moves to block B1160. In block B1160, the imaging station 100 increases N by 1, and the flow then returns to block B1120. If the imaging station 100 determines that blocks B1120-B1150 have been performed for every frame (N=Ntotal) (B1155=Yes), then the flow ends in block B1165.

Furthermore, some guide wires and guide catheters cause an artifact to appear along their center lines along their longitudinal axes. In a graph that shows the differences between the respective sums of neighboring angles for the angles (e.g., the graph in FIG. 13B), such artifacts cause another peak to appear between the peaks of the step-down angle θ_Lo and the step-up angle θ_High. Thus, in some embodiments, in block B1135 the imaging station 100 also determines whether such a peak exists between the step-down angle θ_Lo and the step-up angle θ_High. If such a peak exists and the step-down angle θ_Lo and the step-up angle θ_High are within θmax, then the flow moves to block B1140. If either such a peak does not exist or the step-down angle θ_Lo and the step-up angle θ_High are not within θmax, then the flow moves to block B1145.

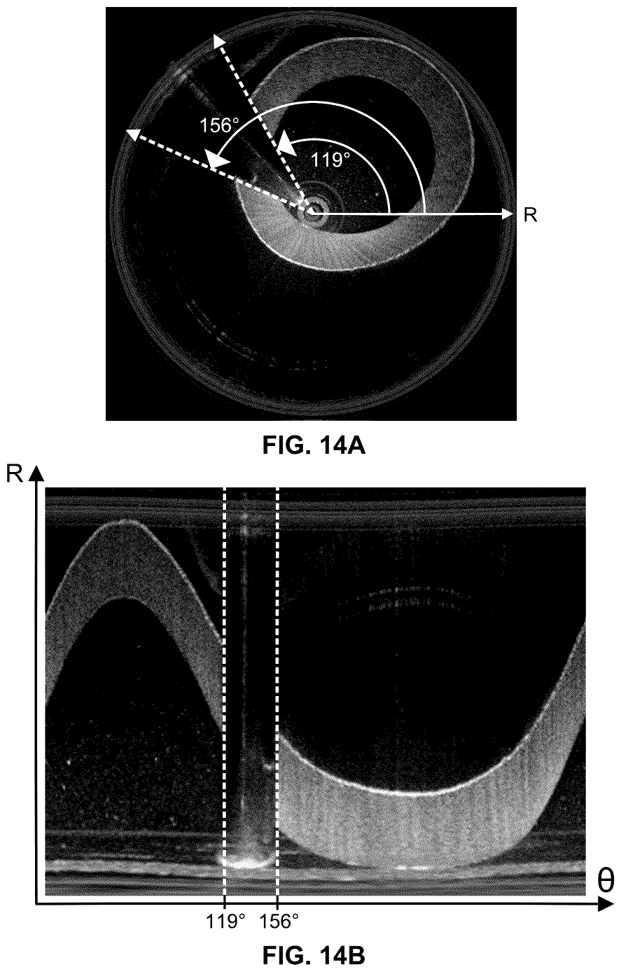

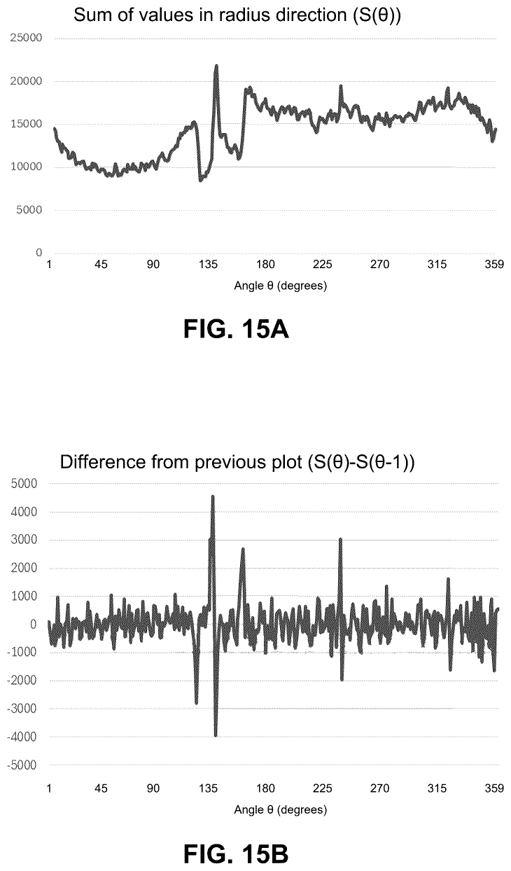

Also for example, FIG. 14A illustrates an example embodiment of an OCT frame, and FIG. 14B illustrates the OCT frame from FIG. 14A in a different coordinate system. In FIG. 14B, the pixels are arranged such that the lower-left corner indicates an angle of 0° and a radial distance R of 0. FIG. 15A illustrates the respective sums of the pixel values in the OCT frame that is shown in FIG. 14A. And FIG. 15B shows the differences between the respective sums of neighboring angles for the angles and sums that are shown in FIG. 15A. In FIGS. 15A and 15B, 119° is a step-down angle θ_Lo (an angle that has an abrupt step-down change). Also, 119° is marked in FIGS. 14A and 14B. 156° is a step-up angle θ_High (an angle that has an abrupt step-up change) and is marked in FIGS. 14A and 14B. An artifact is visible from 119° to 156°.

FIG. 16 illustrates an example embodiment of an operational flow for detecting an artifact in fluorescence-detection data. The flow starts in block B1600 and moves to block B1610, where an imaging station 100 generates one or more fluorescence images based on fluorescence-detection data.

Next, in block B1615, the imaging station 100 obtains (e.g., receives, acquires, retrieves) an identifier of a guide wire or a guide catheter. The identifier may indicate a particular model or type of guide wire or guide catheter. The imaging station 100 may receive the identifier through a user input or retrieve the identifier from memory (e.g., memory that stores a setting that indicates the identifier).

Next, in block B1620, the imaging station 100 obtains a mesh pattern that corresponds to the identifier. For example, the imaging station 100 may use the identifier to retrieve the mesh pattern from storage or from another computing device. Some guide wires and guide catheters appear as a mesh pattern in fluorescence images.

Then, in block B1625, the imaging station 100 identifies any areas in the fluorescence image that include the mesh pattern. And, in block B1630, the imaging station 100 records the areas that include the mesh pattern, for example by recording information that identifies the areas that include the mesh pattern in storage. Finally, the flow ends in block B1635.

FIG. 17 illustrates an example embodiment of an operational flow for removing an artifact from fluorescence images. The flow starts in block B1700 and then moves to block B1710, where an imaging station 100 sets a frame index N to 1. Then, in block B1715, the imaging station 100 selects fluorescence frame N. Next, in block B1720, the imaging station 100 obtains the step-down angle θ_Lo and the step-up angle θ_High for the OCT frame that corresponds to fluorescence frame N. The OCT frame that corresponds to fluorescence frame Nis an OCT frame that is co-registered with fluorescence frame N, and consequently both the corresponding OCT frame and fluorescence frame N show the same features (e.g., parts of a sample 601, parts of a guide wire) in the same locations.

Then, in block B1725, in fluorescence frame N, the imaging station 100 sets the values of the pixels that are between the step-down angle θ_Lo and the step-up angle θ_High to zero or another value that is both (1) a valid pixel value (i.e., a pixel value that is valid for display) and (2) a value that indicates that either no fluorescent light was detected or that an invalid fluorescence signal (artifact) was detected at the pixel. Also, the imaging station 100 may set the values of the pixels that satisfy at least one of the following criteria to zero or another value that is both (1) a valid pixel value (i.e., a pixel value that is valid for display) and (2) a value that indicates that either no fluorescent light was detected or that an invalid fluorescence signal (artifact) was detected at the pixel: pixels that are within a predetermined tolerance (e.g., +5°, +10°) of the step-down angle θ_Lo or the step-up angle θ_High; and pixels that are within a predetermined angular span (e.g., 25°, 30°) of the center angle between the step-down angle θ_Lo and the step-up angle θ_High (e.g., if the step-down angle θ_Lo is 60° and the step-up angle θ_High is 90°, then the center angle is) 75°.

The flow then moves to block B1730, where the imaging station 100 determines whether blocks B1720-B1725 have been performed for every fluorescence frame (whether N=Ntotal, where Ntotal is the total number of fluorescence frames). If blocks B1720-B1725 have not been performed for every fluorescence frame (B1730=No), then in block B1735, the imaging station 100 increases N by one, and the flow returns to block B1715.

If blocks B1720-B1725 have been performed for every fluorescence frame (B1730=Yes), then the flow ends in block B1740.

FIG. 18 illustrates an example embodiment of an operational flow for replacing an artifact in fluorescence images. The flow starts in block B1800. Blocks B1710-B1720 and B1730-B1735 are similar or identical to blocks B1710-B1720 and B1730-B1735 from FIG. 17.

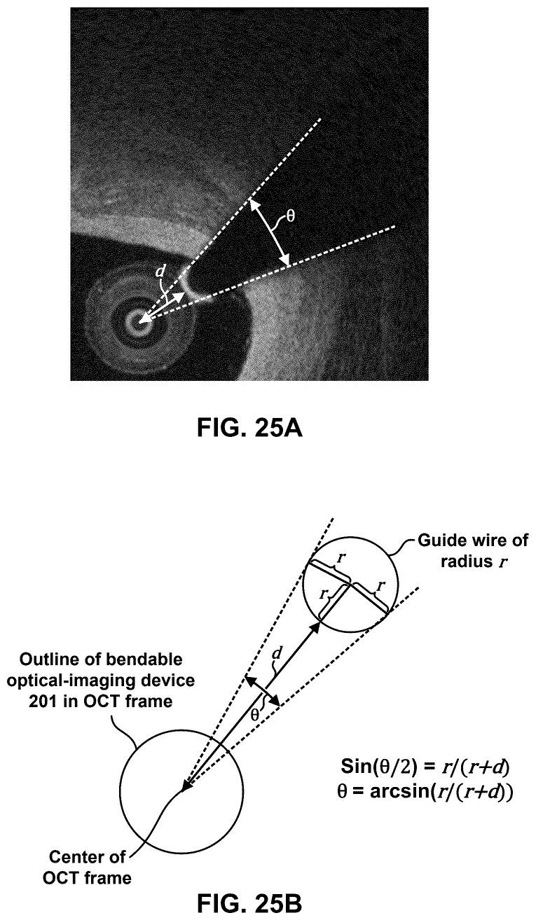

In block B1825, in fluorescence frame N, the imaging station 100 sets the values of the pixels that satisfy at least one of the following criteria to a value that is not a valid pixel value (e.g., −99, null): pixels that are between the step-down angle θ_Lo and the step-up angle θ_High; pixels that are within a predetermined tolerance (e.g., ±5°, ±10°) of the step-down angle θ_Lo or the step-up angle θ_High; and pixels that are within a predetermined angular span (e.g., 25°, 30°) of the center angle between the step-down angle θ_Lo and the step-up angle θ_High. The predetermined angular range may be calculated from the guide wire's size. FIGS. 25A-B illustrate the calculation of a guide wire's angular range. In order to magnify the area of interest in the OCT frame, FIG. 25A shows a partial view of the OCT frame. The angular range may be more accurately calculated for each frame by the guide-wire size and the distance from the guide wire to the center of the frame. The OCT image provides the distance d to the guide-wire surface. Given the guide-wire radius r, the angular extent θ of the guide wire can be described by, or calculated as, the following:

$$θ = 2 arcsin ( r / ( r + d ) ) or θ = 2 {sin}^{- 1} ( \frac{r}{r + d} ) .$$

The distance d to the guide wire may be determined as the minimum distance to the bright line or high intensity border within the angular range between θ_Lo and θ_High, or 201° and 228° in FIG. 12B. The calculation will provide the theoretically accurate angular extent, centered at 214.5°, which is the average of 201° and 228°. This angular range calculation may be used as a cross-check with the determined values of θ_Lo and θ_High.

Once blocks B1720 and B1825 have been performed for every fluorescence frame (B1730=Yes), the flow ends in block B1840.

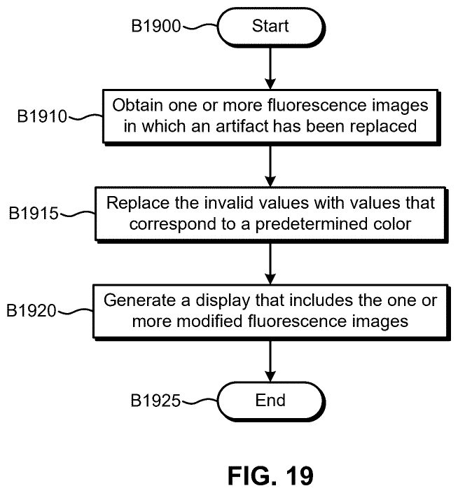

FIG. 19 illustrates an example embodiment of an operational flow for displaying fluorescence images in which an artifact has been replaced.

The flow starts in block B1900, where the imaging station 100 obtains one or more fluorescence images (e.g., fluorescence frames) in which an artifact has been replaced. As described above, an artifact is replaced by changing the values of the pixels that correspond to the artifact to values that are invalid. Next, in block B1910, the imaging station 100 replaces the invalid values with values that correspond to a predetermined color, such as a color that would not otherwise appear in a fluorescence image (e.g., frame). This helps a viewer identify the parts of the fluorescence images that do not show valid fluorescence information, for example because the fluorescence-detection data for these parts was not acquired, was invalid, or was not indicative of the sample 601. The output of block B1910 is one or more modified fluorescence images. Then, in block B1920, the image station 100 generates a display that includes the one or more modified fluorescence images (e.g., fluorescence frames). And the flow ends in block B1925.

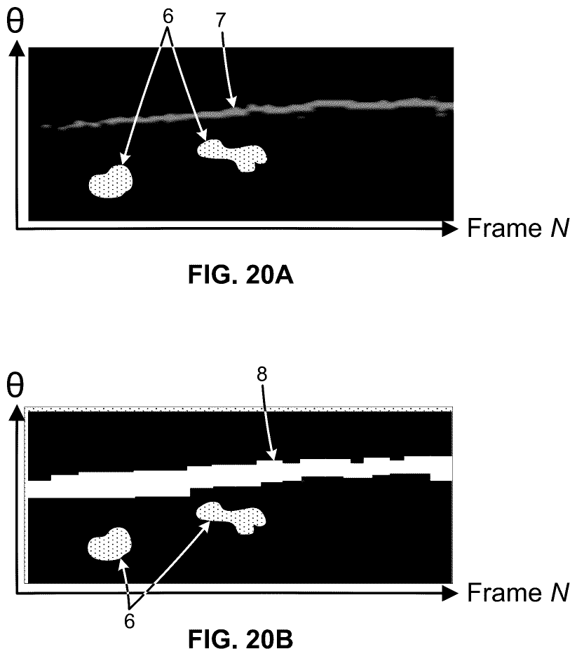

For example, FIG. 20A illustrates fluorescence frames that include an artifact. In FIGS. 20A and 20B, the vertical axis is the angle θ, and the horizontal axis is the frame number N. Each frame may be a column of pixels that is a single pixel wide or a few pixels wide. The fluorescence frames include fluorescence areas 6, which are areas in which fluorescence was detected and in which the pixel values indicated the detected fluorescence. The fluorescence frames also include a guide-wire artifact 7. The values of the pixels that correspond to the guide-wire artifact 7 were replaced with invalid values. FIG. 20B illustrates a display of the fluorescence frames in which the values of the pixels that correspond to the guide-wire artifact 7 were replaced with invalid values. When the fluorescence frames are displayed, the values of the replaced pixels 8 are changed to the value of a predetermined color (e.g., red, pink, orange), for example as described in block B1915 in FIG. 19. Also, the value that is used to represent the color that is displayed may not be the same as the values of the replaced pixels, and thus may not be the same as the specified pixel value that is used when removing or replacing an artifact. For example, if the specified pixel value is-99 (which may not be a pixel value that is valid for display), then the value that is used to represent the color that is displayed may not be the same as −99. This allows a viewer to easily identify the pixels for which the values were replaced and which correspond to an artifact.

FIG. 21 illustrates an example embodiment of a user interface that allows a user to select an area in which to perform artifact detection. The user interface 514 displays fluorescence frames 513. The fluorescence frames 513 include an artifact 7 that was caused by guide catheter and includes fluorescence areas 6. The user interface 514 includes a marker 515. The marker 515 indicates a boundary where artifact detection is to begin or end, and a user may move the marker 515 to control where artifact detection is performed. The imaging station 100 performs artifact detection and artifact removal or performs artifact detection and artifact replacement in the fluorescence frames that are proximal to the marker 515, for example as described in FIG. 6, FIG. 7, FIG. 9, FIG. 10, FIG. 11, or FIG. 16. Also, the frame numbers of the frames that are proximal to the marker 515 are greater than the frame number of the frame at the marker 515.

FIG. 22 illustrates an example embodiment of a user interface that allows a user to select an area in which to perform artifact detection. The user interface 514 displays fluorescence frames 513. The fluorescence frames 513 include an artifact 7 that was caused by guide catheter and includes fluorescence areas 6. The user interface 514 includes a proximal marker 516 and a distal marker 517, and a user may move the proximal marker 516 and the distal marker 517 to control where artifact detection is performed. The imaging station 100 performs artifact detection and artifact removal or performs artifact detection and artifact replacement in the fluorescence frames that are proximal to the proximal marker 516 and in the fluorescence frames that are distal to the distal marker 517, for example as described in FIG. 6, FIG. 7, FIG. 9, FIG. 10, FIG. 11, or FIG. 16. Thus, in such embodiments, the imaging station does not perform artifact detection, artifact removal, or artifact replacement in the fluorescence frames that are between the proximal marker 516 and the distal marker 517. Also, the frame numbers of the frames that are proximal to the proximal maker 517 are greater than the frame number of the frame at the proximal marker 517, and the frame numbers of the frames that are distal to the distal maker 516 are less than the frame number of the frame at the distal marker 516. And the imaging station 100 may identify the fluorescence frames that are proximal to the proximal marker 516 and the fluorescence frames that are distal to the distal marker 517 as being artifact data.

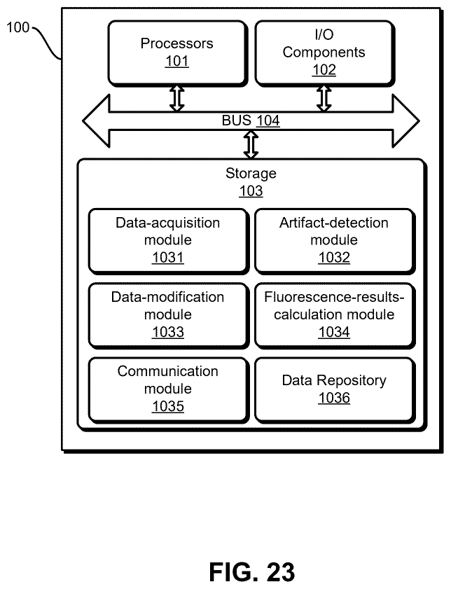

FIG. 23 is a schematic illustration of an example embodiment of an imaging station. The imaging station 100 includes one or more processors 101, one or more computer-readable storage media 103, one or more I/O components 102, and a bus 104.

The one or more processors 101 are or include one or more central processing units (CPUs), such as microprocessors (e.g., a single core microprocessor, a multi-core microprocessor); one or more graphics processing units (GPUs); one or more application-specific integrated circuits (ASICs); one or more field-programmable-gate arrays (FPGAs); one or more digital signal processors (DSPs); or other electronic circuitry (e.g., other integrated circuits). Furthermore, a processor 101 may be a purpose-built controller or may be a general-purpose controller. The one or more processors 101 may include a plurality of processors that include processors that are both (i) included in the imaging station 100 and (ii) in communication with the imaging station 100 but not included in the imaging station 100. And the one or more processors 101 are an example of a processing unit.

The one or more processors 101 may operate based on computer-readable instructions (e.g., in one or more programs) stored on one or more computer-readable storage media 103. As used herein, a computer-readable storage medium 103 includes an article of manufacture, for example a magnetic disk (e.g., a floppy disk, a hard disk), an optical disc (e.g., a CD, a DVD, a Blu-ray), a magneto-optical disk, magnetic tape, and semiconductor memory (e.g., a non-volatile memory card, flash memory, a solid-state drive, SRAM, DRAM, EPROM, EEPROM), and thus a computer-readable storage medium 103 is not a mere transitory, propagating signal. And examples of the one or more computer-readable storage media 103 include networked-attached storage (NAS) devices, intranet-connected storage devices, and internet-connected storage devices. The one or more computer-readable storage media 103, which may include both ROM and RAM, can store computer-readable data or computer-executable instructions. Furthermore, in embodiments where the one or more computer-readable storage media 103 include RAM, the one or more processors 101 can use the RAM as a work area. Additionally, when the imaging station 100 or the one or more processors 101 are described as obtaining information or data, recording information or data, generating information or data, storing information or data, operating on information or data, processing information or data, etc., the information or data are stored in the one or more computer-readable storage media 103. Also, the one or more computer-readable storage media 103 are an example of a storage unit. And the computer-readable storage media 103 may be distributed among multiple processors 101.

The imaging station 100 also includes I/O components 102. The I/O components 102 include physical interfaces and communication components (e.g., a GPU, a network-interface controller) that enable communication (wired or wireless) with other members of a medical-imaging system 10 (e.g., a bendable optical-imaging device 201, a probe-interface subsystem 300, a display device 500), with other computing devices (e.g., a networked computer, a PACS 1400), and with input or output devices, which may include a display device, a network device, a keyboard, a mouse, a printing device, a light pen, an optical-storage device, a scanner, a microphone, a drive, a joystick, and a control pad.

Also, the hardware components of the imaging station 100 communicate via one or more buses 104 or other electrical connections. Examples of buses 104 include a universal serial bus (USB), an IEEE 1394 bus, a PCI bus, an Accelerated Graphics Port (AGP) bus, a Serial AT Attachment (SATA) bus, and a Small Computer System Interface (SCSI) bus.

The imaging station 100 additionally includes a data-acquisition module 1031, an artifact-detection module 1032, a data-modification module 1033, a fluorescence-results-calculation module 1034, and a communication module 1035. As used herein, a module includes logic, computer-readable data, or computer-executable instructions. In the embodiment shown in FIG. 23, the modules are implemented in software (e.g., Assembly, C, C++, C#, Java, JavaScript, BASIC, Perl, Visual Basic, Python, PHP). However, in some embodiments, the modules are implemented in hardware (e.g., customized circuitry) or, alternatively, a combination of software and hardware. When the modules are implemented, at least in part, in software, then the software can be stored in the one or more computer-readable storage media 103. Also, in some embodiments, the imaging station 100 includes additional or fewer modules, the modules are combined into fewer modules, or the modules are divided into more modules. And each of these modules may use (e.g., call) other modules. Also, the imaging station 100 includes a data repository 1036, which stores information, detection data and images.

The data-acquisition module 1031 includes instructions that cause and enable the applicable components (e.g., the one or more processors 101, the storage 103, the I/O components 102) of the imaging station 100 to communicate with and to control the other members of a medical-imaging system 100, such as a probe-interface subsystem 300, and to acquire detection data (which includes fluorescence-detection data and which may include OCT-detection data) from the probe-interface subsystem 300. For example, some embodiments of the data-acquisition module 1031 include instructions that cause the applicable components of the imaging station 100 to control the applicable components of a medical-imaging system 100 to perform at least some of the operations that are described in blocks B610-B620 in FIG. 6, in blocks B610-B620 in FIG. 9, and in blocks B1010 and B620 in FIG. 10. The applicable components of the imaging station 100 operating according to the data-acquisition module 1031 realize an example of a data-acquisition unit.

The artifact-detection module 1032 includes instructions that cause and enable the applicable components (e.g., the one or more processors 101, the storage 103, the I/O components 102) of the imaging station 100 to detect artifacts in fluorescence-detection data or OCT-detection data. For example, some embodiments of the artifact-detection module 1032 include instructions that cause the applicable components of the imaging station 100 to perform at least some of the operations that are described in block B630 in FIG. 6, in block B630 in FIG. 9, in block B1025 in FIG. 10, in blocks B1100-B1165 in FIG. 11, and in blocks B1600-B1635 in FIG. 16. The applicable components of the imaging station 100 operating according to the artifact-detection module 1032 realize an example of an artifact-detection unit.

The data-modification module 1033 includes instructions that cause and enable the applicable components (e.g., the one or more processors 101, the storage 103, the I/O components 102) of the imaging station 100 to remove or replace artifacts in fluorescence-detection data. For example, some embodiments of the data-modification module 1033 include instructions that cause the applicable components of the imaging station 100 to perform at least some of the operations that are described in blocks B640-B650 in FIG. 6; in blocks B700-B760 in FIG. 7A; in blocks B700-B760 in FIG. 7B; in blocks B930, B940, B945, B955, and B960 in FIG. 9; in blocks B1030, B940, B945, B1055, and B960 in FIG. 10; in blocks B1700-B1740 in FIG. 17; and in blocks B1800-B1840 in FIG. 18. The applicable components of the imaging station 100 operating according to the data-modification module 1033 realize an example of a data-modification unit.

The fluorescence-results-calculation module 1034 includes instructions that cause and enable the applicable components (e.g., the one or more processors 101, the storage 103, the I/O components 102) of the imaging station 100 to calculate fluorescence results based on fluorescence-detection data, from which artifacts may have been removed. For example, some embodiments of the fluorescence-results-calculation module 1034 include instructions that cause the applicable components of the imaging station 100 to perform at least some of the operations that are described in block B935 in FIG. 9 and in blocks B1033 and B935 in FIG. 10. The applicable components of the imaging station 100 operating according to the fluorescence-results-calculation module 1034 realize an example of a fluorescence-results-calculation unit.

The communication module 1035 includes instructions that cause the applicable components (e.g., the one or more processors 101, the storage 103, the I/O components 102) of the imaging station 100 to communicate with input devices, output devices, and one or more other computing devices (e.g., a PACS 1400). The communication may include one or more of the following: displaying images and user interfaces on a display device 500, and receiving inputs from input devices (e.g., selections of marker locations, such as the marker 515 in FIG. 21 and the proximal marker 516 and the distal marker 517 in FIG. 22). For example, some embodiments of the communication module 1035 include instructions that cause the applicable components of the imaging station 100 to perform at least some of the operations that are described in block B660 in FIG. 6, in blocks B950 and B965 in FIG. 9, in blocks B950 and B965 in FIG. 10, and in blocks B1900-B1925 in FIG. 19. And the applicable components operating according to the communication module 1035 realize an example of a communication unit.

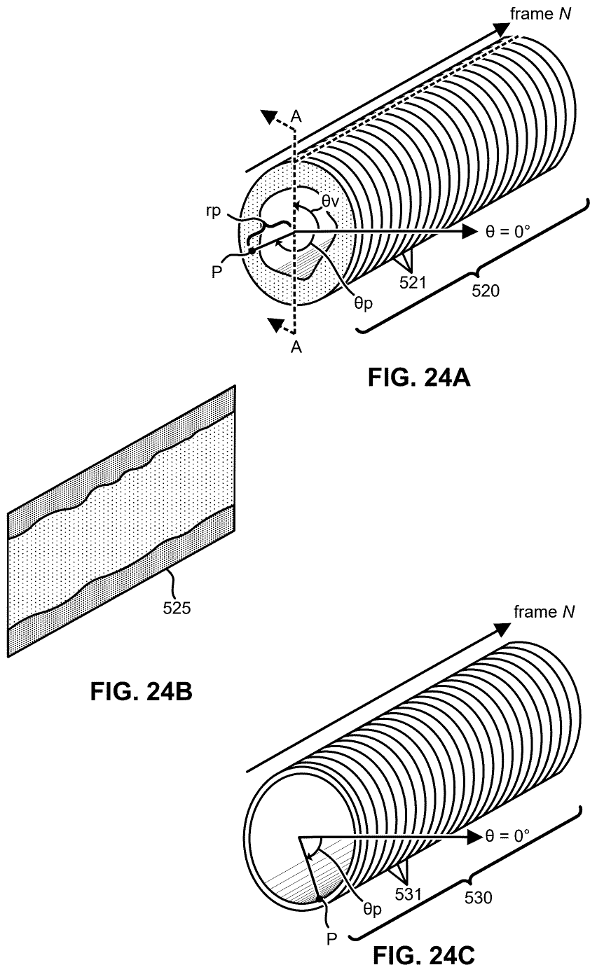

FIG. 24A illustrates an example of a series of tomographic OCT frames of a lumen. The series 520 includes a plurality of tomographic OCT frames 521. Because OCT imaging is a modality that can image structures that are beneath the surface of a sample 601, OCT-detection data may be three dimensional. For example, the OCT-detection data may be defined in a three-dimensional space that is approximately the shape of a cylinder. Thus, collectively, OCT frames 521 (tomographic OCT images) may be defined in a three-dimensional space (as shown in FIG. 24A). Because of this, the location of a pixel P can be defined by the OCT frame 521 that includes the pixel P (or, more generally, by a location on a longitudinal axis), an angle θp (which indicates an angle that is relative to a reference angle that is shown as 0° in FIG. 24A), and a distance rp from a reference location. Accordingly, multiple pixels lie along angle θp (although they have different distances), and multiple pixels are the distance rp from the reference location (although they have different angles).

FIG. 24B illustrates an example a longitudinal view of a lumen that was generated from OCT-detection data. The longitudinal view 525 is a sectional view of the lumen, and the longitudinal view is taken along the plane that is indicated by the line AA in FIG. 24A. Relative to the reference angle, the angle of the plane is Ov. In FIG. 24, the angle θv is 90°. However, a longitudinal view 525 may be taken along a plane that has a different angle (e.g., by a user input of a different angle θv).

FIG. 24C illustrates an example of a series of fluorescence frames of a lumen. The series 530 includes a plurality of fluorescence frames 531. Because fluorescence-detection data is usually two dimensional, each fluorescence frame 531 may be conceptualized as a two-dimensional image that has been wrapped into a cylinder (the height of the cylinder may be only a few pixels wide). Because of this, the location of a pixel P can be defined by the fluorescence frame 531 that includes the pixel P (or, more generally, by a location on a longitudinal axis) and an angle θp (which indicates an angle that is relative to a reference angle that is shown as 0° in FIG. 24C). However, along the angle θp, the location of the pixel is not further defined by a distance because no two pixels in the n-th fluorescence frame 531 can have the same angle θp. Furthermore, in a carpet view, the fluorescence frames 531 may be “unrolled” into planar frames, for example as shown by the third image 511C in FIG. 5.

The data set of a carpet view can be expressed in various arrangements. In some embodiments (for example an example embodiment where there are 400 frames in a pullback and 500 data points in one frame (or one rotation of the imaging core 205 of the catheter 201)), the fluorescence data can be expressed as a matrix F of m rows by n columns, where the row numbers correspond to frame numbers and where the column numbers correspond to the number of detection points (pixels) in one frame (one tomographic view or one rotation of the imaging core 205 of the catheter 201), with its elements expressed with f_{i,j}:

$$F = [ \begin{matrix}{f}_{1 , 1} & {f}_{1 , 2} & ⋯ & {f}_{1 , n} \\ {f}_{2 , 1} & {f}_{2 , 2} & ⋯ & {f}_{2 , n} \\ ⋮ & ⋮ & ⋱ & ⋮ \\ {f}_{m , 1} & {f}_{m , 2} & ⋯ & {f}_{m , n}\end{matrix} ] . \qquad ( 5 )$$

In such embodiments, when an artifact is identified on the point (pixel) corresponding to f_{i,j}, that value of that point (pixel) is replaced by a specified pixel value, such as −99 or 0. Also, the signal on that point (pixel) can be removed by using N/A or null in the memory for this fluorescence-data matrix F, as long as the memory and the imaging station 100 can handle such an entry.

Furthermore, in some embodiments, the fluorescence data may be a data listing as follows:

$$( 1 , 1 , {f}_{11} ) , ( 1 , 2 , {f}_{12} ) , … ( 1 , n , {f}_{1 n} ) ,$$

$$( 2 , 1 , {f}_{21} ) , ( 2 , 2 , {f}_{22} ) , … ( 2 , n , {f}_{2 n} ) ,$$

$$⋯$$

$$( m , 1 , {f}_{m 1} ) , ( m , 2 , {f}_{m 2} ) , … ( m , n , {f}_{mn} ) ,$$

In such data expression, an artifact data may be removed by removing one of data of (i, j, fᵢⱼ). Or replacing the artifact data may be performed by replacing the value of fᵢⱼ with a specified pixel value, such as −99 or 0.

At least some of the above-described devices, systems, and methods can be implemented, at least in part, by providing one or more computer-readable media that contain computer-executable instructions for realizing the above-described operations to one or more computing devices that are configured to read and execute the computer-executable instructions. The systems or devices perform the operations of the above-described embodiments when executing the computer-executable instructions. Also, an operating system on the one or more systems or devices may implement at least some of the operations of the above-described embodiments.

Furthermore, some embodiments use one or more functional units to implement the above-described devices, systems, and methods. The functional units may be implemented in only hardware (e.g., customized circuitry) or in a combination of software and hardware (e.g., a microprocessor that executes software).

In the description, specific details are set forth in order to provide a thorough understanding of the embodiments disclosed. However, well-known methods, procedures, components and circuits may not have been described in detail in order to avoid unnecessarily lengthening the present disclosure.

Also, if a member (e.g., element, part, component) is referred herein as being “on,” “against,” “connected to,” or “coupled to” another member, then the member can be directly on, against, connected or coupled to the other member, but intervening members may also be present between the member and the other member. In contrast, if a member is referred to as being “directly on,” “directly against,” “directly connected to,” or “directly coupled to” another member, then there are no intervening members present between the member and the other member.

Furthermore, the terms “comprising,” “having,” “includes,” “including,” and “containing” are to be construed as open-ended terms unless otherwise noted. Accordingly, these terms, when used in the present specification, specify the presence of described features, integers, steps, operations, elements, materials, or members, but do not preclude the presence or addition of one or more other features, integers, steps, operations, elements, materials, or members that are not explicitly described.

All features disclosed in the specification, including the claims, abstract, and drawings, and all the steps in any method or process disclosed, may be combined in any combination, except combinations where at least some of such features or steps are mutually exclusive.

Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the detailed description.

1. A medical-imaging method comprising:
obtaining imaging data, wherein the imaging data include at least optical-coherence-tomography-imaging (OCT-imaging) data and fluorescence-imaging data;
detecting artifact data in the fluorescence-imaging data based on the OCT-imaging data;
generating modified fluorescence-imaging data by removing the artifact data in the fluorescence-imaging data or by changing the artifact data in the fluorescence-imaging data to a specified value;
generating an image based on the modified fluorescence-imaging data; and
displaying the image based on the modified fluorescence-imaging data and an image based on the OCT-imaging data on a display.

2. (canceled)

3. The medical-imaging method of claim 1,
wherein detecting the artifact data includes:
detecting a first angular position that includes respective OCT-imaging values that change more than a first threshold;
detecting a second angular position that includes respective OCT-imaging values that change more than a second threshold; and
defining the detected range between first angular position and second angular position as the artifact data.

4. The medical-imaging method of claim 1,
wherein the artifact data is detected further based on information of a structure that is depicted in the fluorescence-imaging data.

5. The medical-imaging method of claim 4, wherein the structure is a guide wire or a guide catheter.

6. The medical-imaging method of claim 1, wherein generating the modified fluorescence-imaging data further includes changing the fluorescence-imaging data that are within a predetermined neighboring range of the artifact data to the specified value.

7. The medical-imaging method of claim 1, wherein the specified value is zero or null.

8. The medical-imaging method of claim 1, wherein regions of the image that correspond to the artifact data are shown in a predetermined color.

9. The medical-imaging method of claim 1, further comprising:
performing an analysis of the modified fluorescence-imaging data.

10. The medical-imaging method of claim 1, further comprising:
(i) obtaining a selection of a distal frame of fluorescence-imaging data and specifying frames of the fluorescence-imaging data that have frame numbers that are less than a frame number of the distal frame as being artifact data; or
(ii) obtaining a selection of a proximal frame of fluorescence-imaging data and specifying frames of the fluorescence-imaging data that have frame numbers that are greater than a frame number of the proximal frame as being artifact data.

11. A medical-imaging device comprising:
at least one processor; and
at least one computer-readable storage media that is in communication with the at least one processor, wherein the at least one computer-readable storage media stores instructions for causing the at least one processor and the at least one computer-readable storage media to:
obtain imaging data, wherein the imaging data include at least optical-coherence-tomography-imaging (OCT-imaging) data and fluorescence-imaging data;
detect artifact data in the fluorescence-imaging data based on the OCT-imaging data;
generate modified fluorescence-imaging data by removing the artifact data in the fluorescence-imaging data or by changing the artifact data in the fluorescence-imaging data to a specified value;
generate an image based on the modified fluorescence-imaging data; and
display the image based on the modified fluorescence-imaging data and an image based on the OCT-imaging data on a display.

12. (canceled)

13. The medical-imaging device of claim 11, wherein, to detect the artifact data, the instructions further cause the at least one processor and the at least one computer-readable storage media to:
detect a first angular position that includes respective OCT-imaging values that change more than a first threshold;
detect a second angular position that includes respective OCT-imaging values that change more than a second threshold; and
define the detected range between first angular position and second angular position as the artifact data.

14. The medical-imaging device of claim 11, wherein the instructions further cause the at least one processor and the at least one computer-readable storage media to:
detect the artifact data further based on information of a structure that is depicted in the fluorescence-imaging data.

15. The medical-imaging device of claim 11, wherein the at least one computer-readable storage media further stores instructions for causing the at least one processor and the at least one computer-readable storage media to:
change the fluorescence-imaging data that are within a predetermined neighboring range of the artifact data to the specified value.

16. The medical-imaging device of claim 11, wherein the specified value is zero or null.

17. The medical-imaging device of claim 11, wherein regions of the image that correspond to the artifact data are shown in a predetermined color.

18. The medical-imaging device of claim 11, wherein the at least one computer-readable storage media further stores instructions for causing the at least one processor and the at least one computer-readable storage media to:
perform an analysis of the modified fluorescence-imaging data.

19. A medical-imaging system comprising:
a probe-interface subsystem;
a patient-interface unit;
a bendable optical-imaging device;
at least one processor; and
at least one computer-readable storage media that is in communication with the at least one processor, wherein the at least one computer-readable storage media stores instructions for causing the at least one processor and the at least one computer-readable storage media to:
obtain imaging data, wherein the imaging data include at least optical-coherence-tomography-imaging (OCT-imaging) data and fluorescence-imaging data;
detect artifact data in the fluorescence-imaging data based on the OCT-imaging data;
generate modified fluorescence-imaging data by removing the artifact data in the fluorescence-imaging data or by changing the artifact data in the fluorescence-imaging data to a specified value;
generate an image based on the modified fluorescence-imaging data; and
display the image based on the modified fluorescence-imaging data and an image based on the OCT-imaging data on a display.

20. The medical-imaging system of claim 19, wherein the probe-interface subsystem, the patient-interface unit, and the bendable optical-imaging device are configured to perform both optical-coherence-tomography imaging and fluorescence imaging.
